(19) United States
(12) Patent Application Publication
(10) Pub. No.: US 2024/0426832 A1
(43) Pub. Date: Dec. 26, 2024

(54) METHODS OF IDENTIFYING COMPOUNDS FOR INDUCING PROTEIN-PROTEIN INTERACTION

(71) Applicant: Oncopia Therapeutics, Inc. d/b/a/ SK Life Science Labs, New York, NY (US)

(72) Inventors: Bomie Han, King of Prussia, PA (US); Corey Ohman Strickland, King of Prussia, PA (US)

(21) Appl. No.: 18/339,429

(22) Filed: Jun. 22, 2023

(51) Int. Cl. G01N 33/68; G01N 33/58

(52) U.S. Cl. CPC G01N 33/6845; G01N 33/582

(57) ABSTRACT

The present disclosure relates method of identifying a compound targeting a first protein and a second protein, wherein the first protein and/or the second protein are associated with a disease or disorder.

### BACKGROUND

Molecular glues are chemical compounds that cause an induced proximity of one or more proteins to each other. The molecular glue compounds enhance the affinity of one or more proteins to each other, thus causing the proteins to interact. Depending on the specific protein(s), the interaction can impact the function of one or both proteins. The functional consequences can include, but are not limited to: changes in protein stability, changes in protein levels, changes in protein post-translational modification, changes in protein localization, and/or changes in protein activity.

In the context of protein degradation, a molecular glue enhances the binding of a protein of interest (POI) to an E3 ligase (E3), which in turn ultimately leads to the Ubiquitin Proteasome System (UPS)-mediated degradation of the POI. Molecular glues are challenging to discover for several reasons. First, they can often show little or no binding to either of the individual POI or E3. It is often only in the presence of the molecular glue, POI and E3 that a complex is observed. Even when a glue compound shows measurable binding to either protein, it is the ternary complex that matters and most binary binding events do not lead to ternary complex formation. Therefore, all three components, POI, E3, and molecular glue need to be present during the screen. Secondly, the requirement of presence of all three components in the screening assay mixture poses an enormous challenge since it is extremely difficult to predict which of the large number (>600) of E3 ligases in the human proteome is likely to form a ternary complex with a given POI.

As a large number of compounds need to be screened, often exceeding hundreds of thousands, testing many individual E3 ligases in combination with the large number of compounds becomes challenging. There thus remains a need for novel methods of identifying molecular glues.

### SUMMARY

In some aspects, the present disclosure provides a method of identifying a first protein, a second protein, a compound targeting the first protein and the second protein, or any combination thereof.

In some aspects, the present disclosure provides a first protein identified by the method of the present disclosure.

In some aspects, the present disclosure provides a second protein identified by the method of the present disclosure.

In some aspects, the present disclosure provides a combination of a first protein and a second protein, identified by the method of the present disclosure.

In some aspects, the present disclosure provides a combination of a first protein, a second protein, and a compound targeting the first protein and the second protein, identified by the method of the present disclosure.

In some aspects, the present disclosure provides a compound identified by the method of the present disclosure.

In some aspects, the present disclosure provides a method of degrading a first protein in a subject, comprising administering to the subject a compound identified by the method of the present disclosure.

In some aspects, the present disclosure provides a compound identified by the method of the present disclosure, for use in degrading a first protein in a subject.

In some aspects, the present disclosure provides a use of a compound identified by the present disclosure in the manufacture of a medicament for degrading a first protein in a subject.

In some aspects, the present disclosure provides a method of treating and/or preventing a disease or disorder associated with a first protein in a subject, comprising administering to the subject a therapeutically effective amount of a compound identified by the method of the present disclosure.

In some aspects, the present disclosure provides a compound identified by the method of the present disclosure for use in treating and/or preventing a disease or disorder associated with a first protein in a subject.

In some aspects, the present disclosure provides a use of a compound identified by the method of the present disclosure in the manufacture of a medicament for treating and/or preventing a disease or disorder associated with a first protein in a subject.

In some aspects, the present disclosure provides a method of degrading a second protein in a subject, comprising administering to the subject a compound identified by the method of the present disclosure.

In some aspects, the present disclosure provides a compound identified by the method of the present disclosure, for use in degrading a second protein in a subject.

In some aspects, the present disclosure provides a use of a compound identified by the present disclosure in the manufacture of a medicament for degrading a second protein in a subject.

In some aspects, the present disclosure provides a method of treating and/or preventing a disease or disorder associated with a second protein in a subject, comprising administering to the subject a therapeutically effective amount of a compound identified by the method of the present disclosure.

In some aspects, the present disclosure provides a compound identified by the method of the present disclosure for use in treating and/or preventing a disease or disorder associated with a second protein in a subject.

In some aspects, the present disclosure provides a use of a compound identified by the method of the present disclosure in the manufacture of a medicament for treating and/or preventing a disease or disorder associated with a second protein in a subject.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTION OF THE DRAWINGS

FIG. 1A is a graph showing the simulated time-course and dose-dependence of fluorescence signal development for GFP11-RBM39 and GFP10-DCAF15 (Combination A).

FIG. 1B is a graph showing the simulated time-course and dose-dependence of fluorescence signal development for RBM39-GFP11 and GFP10-DCAF15 (Combination B).

FIG. 1C is a graph showing the simulated time-course and dose-dependence of fluorescence signal development for GFP11-RBM39 and DCAF15-GFP10 (Combination C).

FIG. 1D is a graph showing the simulated time-course and dose-dependence of fluorescence signal development for RBM39-GFP11 and DCAF15-GFP10 (Combination D).

FIG. 2A is a graph showing the simulated effect of GFP-booster on fluorescence signal intensity for Combination A and Combination C.

FIG. 2B is a graph showing the simulated effect of GFP-booster on fluorescence signal intensity for Combination B and Combination D.

FIG. 3 is a simulated graph showing the potential detection of test compounds from MALDI MS experiment, based on the simulated MS signal between indisulam and lenalidomide.

FIG. 4 is a simulated graph showing the potential detection of E3 from MALDI MS experiment.

FIG. 5 is a graph with experimental data showing the time-course of fluorescence signal development for IKZF2-CRBN Glue Compound 1, IKZF2-CRBN Glue Compound 2, IKZF2-CRBN Glue Compound 3 and IKZF2-CRBN Non-Glue compounds Chloroquinoxaline and Indisulam.

### DETAILED DESCRIPTION

The present disclosure relates to methods of identifying compounds for inducing protein-protein interaction. The present disclosure also relates to the compounds identified in the methods, and uses of the compounds, e.g., in degrading POIs, and/or treating or preventing diseases or disorders. The methods of this disclosure are applicable to various fields, including but not limited to human therapy, agriculture and animal health.

### Methods of the Present Disclosure

In some aspects, the present disclosure provides a method of identifying a first protein, a second protein, a compound targeting the first protein and the second protein, or any combination thereof.

In some embodiments, the method is of identifying a first protein (e.g., a POI) that is targeted by a compound with a second protein.

In some embodiments, the method is of identifying a second protein that is targeted by a compound with a first protein.

In some embodiments, the method is of identifying a combination of a first protein and a second protein that is targeted by a compound.

In some embodiments, the method is of identifying a combination of a first protein, a second protein, and a compound targeting the first protein and the second protein.

In some embodiments, the method comprises:
(i) providing an assay mixture comprising:
   (a) a first protein or fragment thereof, covalently attached to a first tag fragment;
   (b) a second protein or a fragment thereof, covalently attached to a second tag fragment, wherein the second tag fragment is complementary to the first tag fragment; and
   (c) a candidate compound,
   wherein the first tag fragment and the second tag fragment are configured to generate or enhance an assay signal upon the assay mixture resulting in an induced proximity between the first protein or fragment thereof, and the second protein or fragment thereof.

In some embodiments, the method comprises:
(i) providing an assay mixture comprising:
   (a) a first protein or a fragment thereof, covalently attached to a first tag fragment;
   (b) a second protein or a fragment thereof, covalently attached to a second tag fragment, wherein the second tag fragment is complementary to the first tag fragment; and
   (c) a candidate compound,
   wherein the first protein or fragment thereof, and/or the second protein or fragment thereof, is covalently attached to an affinity component; and
   wherein the first tag fragment and the second tag fragment are configured to generate or enhance an assay signal upon the assay mixture resulting in a complex comprising the first protein or fragment thereof, the second protein or fragment thereof, and the compound;
(ii-a) contacting the complex with an immobilized affinity binder targeting the affinity component, thereby forming an immobilized complex; and
(ii-b) detecting and/or isolating the immobilized complex, thereby identifying the first protein, the second protein, the compound, or any combination thereof.

In some embodiments, the method comprises:
(i) providing an assay mixture comprising:
   (a) a first protein or fragment thereof, covalently attached to a first tag fragment;
   (b) a second protein or a fragment thereof, covalently attached to a second tag fragment, wherein the second tag fragment is complementary to the first tag fragment; and
   (c) a candidate compound,
   wherein the assay mixture results in an induced proximity between the first protein or fragment thereof, and the second protein or fragment thereof; such that the first tag fragment and the second tag fragment generate or enhance an assay signal.

In some embodiments, the method comprises:
(i) providing an assay mixture comprising:
   (a) a first protein or a fragment thereof, covalently attached to a first tag fragment;
   (b) a second protein or a fragment thereof, covalently attached to a second tag fragment, wherein the second tag fragment is complementary to the first tag fragment; and
   (c) a candidate compound,
   wherein the first tag fragment and the second tag fragment are configured to generate or enhance an assay signal upon the assay mixture resulting in a complex comprising the first protein or fragment thereof, the second protein or fragment thereof, and the compound; and
(ii) identifying the first protein, the second protein, the compound, or any combination thereof associated with the complex.

In some embodiments, the method comprises:
(i) providing an assay mixture comprising:
   (a) a first protein or a fragment thereof, covalently attached to a first tag fragment;
   (b) a second protein or a fragment thereof, covalently attached to a second tag fragment, wherein the second tag fragment is complementary to the first tag fragment; and
   (c) a candidate compound,
   wherein the first protein or fragment thereof, and/or the second protein or fragment thereof, is covalently attached to an affinity component; and
   wherein the assay mixture results in a complex comprising the first protein or fragment thereof, the second protein or fragment thereof, and the compound; such that the first tag fragment and the second tag fragment generate or enhance an assay signal;
(ii-a) contacting the complex with an immobilized affinity binder targeting the affinity component, thereby forming an immobilized complex; and
(ii-b) detecting and/or isolating the immobilized complex, thereby identifying the first protein, the second protein, the compound, or any combination thereof.

In some embodiments, the method comprises:
(i) providing an assay mixture comprising:
   (a) a first protein or a fragment thereof, covalently attached to a first tag fragment;
   (b) a second protein or a fragment thereof, covalently attached to a second tag fragment, wherein the second tag fragment is complementary to the first tag fragment; and
   (c) a candidate compound,
   wherein the first tag fragment and the second tag fragment are configured to generate or enhance an assay signal upon the assay mixture resulting in an induced proximity between the first protein or fragment thereof, and the second protein or fragment thereof; and
(ii) identifying the first protein, the second protein, the compound, of any combination thereof associated with (e.g., causing) the induced proximity;
   wherein the identified compound is capable of causing the degradation of the first protein (e.g., the POI), in the presence of the second protein (e.g., the E3 ligase).

In some embodiments, the induced proximity is associated with (e.g., caused by) the compound.

In some embodiments, the assay mixture further comprises a detector fragment complementary to the first tag fragment and the second tag fragment.

In some embodiments, the first tag fragment, the second tag fragment, and the detector fragment are configured to generate or enhance an assay signal upon the assay mixture resulting in an induced proximity between the first protein or fragment thereof, and the second protein or fragment thereof.

In some embodiments, the method comprises:
(i) providing an assay mixture of:
   (a) a first protein or a fragment thereof, covalently attached to a first tag fragment;
   (b) a second protein or a fragment thereof, covalently attached to a second tag fragment;
   (c) a detector fragment complementary to the first tag fragment and the second tag fragment; and
   (d) a candidate compound,
   wherein the first tag fragment, the second tag fragment, and the detector fragment are configured to generate or enhance an assay signal upon the assay mixture resulting in an induced proximity between the first protein or fragment thereof, and the second protein or fragment thereof.

In some embodiments, the method comprises:
(i) providing an assay mixture of:
   (a) a first protein or a fragment thereof, covalently attached to a first tag fragment;
   (b) a second protein or a fragment thereof, covalently attached to a second tag fragment;
   (c) a detector fragment complementary to the first tag fragment and the second tag fragment; and
   (d) a candidate compound,
   wherein the first protein or fragment thereof, and/or the second protein or fragment thereof, is covalently attached to an affinity component; and
   wherein the first tag fragment, the second tag fragment, and the detector fragment are configured to generate or enhance an assay signal upon the assay mixture resulting in an induced proximity between the first protein or fragment thereof, and the second protein or fragment thereof;
(ii-a) contacting the complex with an immobilized affinity binder targeting the affinity component, thereby forming an immobilized complex; and
(ii-b) detecting and/or isolating the immobilized complex, thereby identifying the first protein, the second protein, the compound, or any combination thereof.

In some embodiments, the method comprises:
(i) providing an assay mixture of:
   (a) a first protein or a fragment thereof, covalently attached to a first tag fragment;
   (b) a second protein or a fragment thereof, covalently attached to a second tag fragment;
   (c) a detector fragment complementary to the first tag fragment and the second tag fragment; and
   (d) a candidate compound,
   wherein the first tag fragment, the second tag fragment, and the detector fragment are configured to generate or enhance an assay signal upon the assay mixture resulting in an induced proximity between the first protein or fragment thereof, and the second protein or fragment thereof; and
   wherein the identified compound is capable of causing the degradation of the first protein (e.g., the POI), in the presence of the second protein (e.g., the E3 ligase).

In some embodiments, the method comprises:
(i) providing an assay mixture of:
   (a) a first protein or a fragment thereof, covalently attached to a first tag fragment;
   (b) a second protein or a fragment thereof, covalently attached to a second tag fragment;
   (c) a detector fragment complementary to the first tag fragment and the second tag fragment; and
   (d) a candidate compound,
   wherein the assay mixture results in an induced proximity between the first protein or fragment thereof, and the second protein or fragment thereof; such that the first tag fragment, the second tag fragment, and the detector fragment are configured to generate or enhance an assay signal.

In some embodiments, the method comprises:
(i) providing an assay mixture of:
   (a) a first protein or a fragment thereof, covalently attached to a first tag fragment;
   (b) a second protein or a fragment thereof, covalently attached to a second tag fragment;
   (c) a detector fragment complementary to the first tag fragment and the second tag fragment; and
   (d) a candidate compound,
   wherein the first protein or fragment thereof, and/or the second protein or fragment thereof, is covalently attached to an affinity component; and
   wherein the assay mixture results in an induced proximity between the first protein or fragment thereof, and the second protein or fragment thereof; such that the first tag fragment, the second tag fragment, and the detector fragment are configured to generate or enhance an assay signal;
(ii-a) contacting the complex with an immobilized affinity binder targeting the affinity component, thereby forming an immobilized complex; and
(ii-b) detecting and/or isolating the immobilized complex, thereby identifying the first protein, the second protein, the compound, or any combination thereof.

In some embodiments, the method comprises:
(i) providing an assay mixture of:
   (a) a first protein or a fragment thereof, covalently attached to a first tag fragment;
   (b) a second protein or a fragment thereof, covalently attached to a second tag fragment;
   (c) a detector fragment complementary to the first tag fragment and the second tag fragment; and
   (d) a candidate compound,
   wherein the assay mixture results in an induced proximity between the first protein or fragment thereof, and the second protein or fragment thereof; such that the first tag fragment, the second tag fragment, and the detector fragment are configured to generate or enhance an assay signal; and
   wherein the identified compound is capable of causing the degradation of the first protein (e.g., the POI), in the presence of the second protein (e.g., the E3 ligase).

In some embodiments, the assay mixture comprises a plurality of different first proteins or fragments thereof.

In some embodiments, the assay mixture results in the complex such that the assay signal is generated or enhanced.

In some embodiments, the assay mixture results in induced proximity among protein molecules such that the assay signal is generated or enhanced.

In some embodiments, the assay mixture comprises at least 2, at least 5, at least 10, at least 20, at least 30, at least 40, at least 50, at least 100, at least 200, at least 300, at least 400, at least 500, at least 600, at least 700, at least 800, at least 900, or at least 1000 different first proteins or fragments thereof.

In some embodiments, the assay mixture comprises from about 1 to about 5, from about 5 to about 10, from about 10 to about 15, from about 15 to about 20, from about 20 to about 25, or from about 25 to about 30, from about 30 to about 35, from about 35 to about 40, from about 40 to about 45, or from about 45 to about 50 different first proteins or fragments thereof.

In some embodiments, the assay mixture comprises from about 50 to about 100, about 100 to about 200, from about 200 to about 300, from about 300 to about 400, from about 400 to about 500, from about 500 to about 600, or from about 600 to about 700 different first proteins or fragments thereof.

In some embodiments, the first protein is an isolated first protein.

In some embodiments, the assay mixture comprises a plurality of different second proteins or fragments thereof.

In some embodiments, the assay mixture comprises at least 2, at least 5, at least 10, at least 20, at least 30, at least 40, at least 50, at least 100, at least 200, at least 300, at least 400, at least 500, or at least 1000 different second proteins or fragments thereof.

In some embodiments, the assay mixture comprises from about 1 to about 5, from about 5 to about 10, from about 10 to about 15, from about 15 to about 20, from about 20 to about 25, or from about 25 to about 30, from about 30 to about 35, from about 35 to about 40, from about 40 to about 45, or from about 45 to about 50 different second proteins or fragments thereof.

In some embodiments, the assay mixture comprises from about 2 to about 5, from about 5 to about 10, or from about 10 to about 20 different second proteins or fragments thereof.

In some embodiments, the assay mixture comprises a plurality of different candidate compounds.

In some embodiments, the assay mixture comprises at least 2, at least 5, at least 10, at least 20, at least 30, at least 40, at least 50, at least 100, at least 200, at least 300, at least 400, at least 500, or at least 1000 different candidate compounds.

In some embodiments, the assay mixture comprises from about 50 to about 250 different candidate compounds.

In some embodiments, the assay mixture comprises from about 250 to about 2500 different candidate compounds.

In some embodiments, the assay mixture comprises more than about 2500 different candidate compounds.

In some embodiments, the first tag fragment and the second tag fragment are configured to generate or enhance an assay signal upon the assay mixture resulting in a complex comprising the first protein or fragment thereof, the second protein or fragment thereof, and the compound.

In some embodiments, the assay mixture results in a complex comprising the first protein or fragment thereof, the second protein or fragment thereof, and the compound; such that the first tag fragment and the second tag fragment generate or enhance an assay signal.

In some embodiments, the first tag fragment, the second tag fragment, and the detector fragment are configured to generate or enhance an assay signal upon the assay mixture resulting in a complex comprising the first protein or fragment thereof, the second protein or fragment thereof, and the compound.

In some embodiments, the assay mixture results in a complex comprising the first protein or fragment thereof, the second protein or fragment thereof, and the compound; such that the first tag fragment, the second tag fragment, and the detector fragment generate or enhance an assay signal.

In some embodiments, the assay mixture results in a complex comprising the first protein or fragment thereof, the second protein or fragment thereof, and the compound.

In some embodiments, the assay signal is a light emission.

In some embodiments, the assay signal is fluorescence, fluorescence polarization, time-resolved fluorescence (TRF), homogenous time resolved fluorescence, luminescence, or UV/Vis.

In some embodiments, the assay signal is fluorescence, luminescence, phosphorescence, or scintillation.

In some embodiments, the assay signal is fluorescence.

In some embodiments, the assay signal is a radioactivity-based signal such as a scintillation-proximity-assay (SPA) signal.

In some embodiments, the assay signal is a radiometric signal.

In some embodiments, the assay signal is a change in color.

In some embodiments, step (i) further comprises detecting the generated or enhanced assay signal (e.g., the fluorescence).

In some embodiments, the methods of the present disclosure are performed one or more times. In some embodiments, the methods of the present disclosure are performed one, two, three, four or five times. In some aspects, the methods of the present disclosure are performed multiple times. In some aspects, the methods of the present disclosure are performed multiple times, wherein the method is performed using new wells each time.

### Tag and Detector Fragments

In some embodiments, the first tag fragment and the second tag fragment are configured to form a signal protein upon the assay mixture resulting in an induced proximity between the first protein or fragment thereof, and the second protein or fragment thereof.

In some embodiments, the assay mixture results in an induced proximity between the first protein or fragment thereof, and the second protein or fragment thereof; such that the first tag fragment and the second tag fragment generate or enhance an assay signal.

In some embodiments, the first tag fragment, the second tag fragment, and the detector fragment are configured to form a signal protein upon the assay mixture resulting in an induced proximity between the first protein or fragment thereof, and the second protein or fragment thereof.

In some embodiments, the assay mixture results in an induced proximity between the first protein or fragment thereof, and the second protein or fragment thereof; such that the first tag fragment, the second tag fragment, and the detector fragment generate or enhance an assay signal.

In some embodiments, the signal protein is a fluorescent protein, e.g., an engineered fluorescent protein.

In some embodiments, the signal protein is a green fluorescent protein (GFP), an enhanced GFP (EGFP), a superfolder GFP (sfGFP), a blue fluorescent protein (e.g., EBFP, EBFP2, Azurite, mKalama1), a cyan fluorescent protein (e.g., ECFP, Cerulean, CyPet, mTurquoise2), a yellow fluorescent protein (e.g., YFP, Citrine, Venus, Ypet), a redox sensitive GFP (roGFP), or a mutant thereof.

In some embodiments, the signal protein is a GFP.

In some embodiments, the signal protein is a GFP mutant (BFPms1), e.g., that preferentially binds Zn (II) and Cu (II) over Ca (II).

In some embodiments, the signal protein is a fluorescent protein (which could be from different natural origin) having the beta barrel structure composed of 11-stranded beta sheets.

In some embodiments, the signal protein is mCherry (which is a member of the mFruits family of monomeric red fluorescent proteins (mRFPs)). In some embodiments, the signal protein is mRFP1, mStrawberry, mOrange, or dTomato.

In some embodiments, the first tag fragment is a first tag green fluorescence protein (GFP) fragment.

In some embodiments, the second tag fragment is a second tag GFP fragment (e.g., a second tag GFP fragment different from the first tag GFP fragment).

In some embodiments, the detector fragment is a detector GFP fragment (e.g., a detector GFP fragment different from the second tag GFP fragment and the first tag GFP fragment).

In some embodiments, the first tag GFP fragment and the second tag GFP fragment are configured to form a GFP, upon the assay mixture resulting in an induced proximity (e.g., induced by the candidate compound) between the first protein or fragment thereof, and the second protein or fragment thereof.

In some embodiments, the assay mixture results in an induced proximity between the first protein or fragment thereof, and the second protein or fragment thereof; such that the first tag GFP fragment and the second tag GFP fragment are configured to form a GFP.

In some embodiments, the first tag GFP fragment, the second tag GFP fragment, and the detector GFP fragment are configured to form a GFP, upon the assay mixture resulting in an induced proximity (e.g., induced by the candidate compound) between the first protein or fragment thereof, and the second protein or fragment thereof.

In some embodiments, the assay mixture results in an induced proximity between the first protein or fragment thereof, and the second protein or fragment thereof; such that the first tag GFP fragment, the second tag GFP fragment, and the detector GFP fragment are configured to form a GFP.

In some embodiments, the first tag GFP fragment and the second tag GFP fragment are independently selected from GFP1, GFP2, GFP3, GFP4, GFP5, GFP6, GFP7, GFP8, GFP9, GFP10, and GFP11. In some embodiments, the first tag GFP fragment and the second tag GFP fragment are selected from the combinations described in Annu. Rev. Biophys. 6(48):19-44 (2019) (incorporated herein by reference).

In some embodiments, the first tag GFP fragment and the second tag GFP fragment are independently selected from GFP1 and GFP2.

In some embodiments, the first tag GFP fragment and the second tag GFP fragment are independently selected from GFP2 and GFP3.

In some embodiments, the first tag GFP fragment and the second tag GFP fragment are independently selected from GFP5 and GFP6.

In some embodiments, the first tag GFP fragment and the second tag GFP fragment are independently selected from GFP7 and GFP8.

In some embodiments, the first tag GFP fragment and the second tag GFP fragment are independently selected from GFP8 and GFP9.

In some embodiments, the first tag GFP fragment and the second tag GFP fragment are independently selected from GFP9 and GFP10.

In some embodiments, the first tag GFP fragment is GFP10, and second tag GFP fragment is GFP11.

In some embodiments, the first tag GFP fragment is GFP11, and second tag GFP fragment is GFP10.

In some embodiments, the detector GFP fragment comprises GFP1, GFP2, GFP3, GFP4, GFP5, GFP6, GFP7, GFP8, and GFP9.

In some embodiments, the detector GFP fragment further comprises a chromophore.

In some embodiments, the first tag GFP fragment, the second tag GFP fragment, and the detector fragment are selected from the combinations described in Table 1 below.

TABLE 1

| First Tag GFP Fragment/Second | |
|---|---|
| Tag GFP Fragment | Detector Fragment* |
| GFP1/GFP2 or GFP2/GFP1 | GFP3, GFP4, GFP5, GFP6, GFP7, |
| | GFP8, GFP9, GFP10, and GFP11 |
| GFP2/GFP3 or GFP3/GFP2 | GFP1, GFP4, GFP5, GFP6, GFP7, |
| | GFP8, GFP9, GFP10, and GFP11 |
| GFP5/GFP6 or GFP6/GFP5 | GFP1, GFP2, GFP3, GFP4, GFP7, |
| | GFP8, GFP9, GFP10, and GFP11 |
| GFP7/GFP8 or GFP8/GFP7 | GFP1, GFP2, GFP3, GFP4, GFP5, |
| | GFP6, GFP9, GFP10, and GFP11 |
| GFP8/GFP9 or GFP9/GFP8 | GFP1, GFP2, GFP3, GFP4, GFP5, |
| | GFP6, GFP7, GFP10, and GFP11 |
| GFP9/GFP10 or GFP10/GFP9 | GFP1, GFP2, GFP3, GFP4, GFP5, |
| | GFP6, GFP7, GFP8, and GFP11 |
| GFP10/GFP11 or GFP11/GFP10 | GFP1, GFP2, GFP3, GFP4, GFP5, |
| | GFP6, GFP7, GFP8, and GFP9 |

*The detector fragment may further comprise a chromophore.

### Targeted Proteins

In some embodiments, the first protein is a protein of interest (POI).

In some embodiments, the fragment of the first protein comprises a binding site of the second protein.

In some embodiments, the fragment of the first protein comprises at least a portion of the binding site of the first protein to the identified compound.

In some embodiments, the fragment of the first protein comprises the binding site of the first protein to the identified compound.

In some embodiments the fragment of the first protein comprises the binding site of the protein of interest.

In some embodiments, the POI is a kinase, a phosphatase, a glycosylase, a deglycosylase, a methylase, a demethylase, a sumoylase, a deubiquitinase, an acetyl transferase, a deacetylase, a fatty acyl transferase, a protease, an isomerase, or an arginase.

In some embodiments, the POI is a cytoskeletal protein or a protein with scaffolding function to bring other proteins together into a complex.

In some embodiments, the POI is a metabolic enzyme, a transcription factor, a cell surface receptor (e.g., a GPCR or a receptor tyrosine kinase), an ion channel, a membrane embedded or associated enzyme (e.g., an adenylyl cyclase), an RNA polymerase, an RNA splicing enzyme, a transporter, a DNA helicase, or a DNA endonuclease.

In some embodiments, the POI is a hydroxylases, a dehyrogenase, a reductase, an oxidase, an oxygenase, an oxidoreductase, a carboxylase, a decarboxylase, a lyase, an aldolase, a desaturase, a mutase, an epimerase, an isomerase, a racemase, an esterase, an amidase, a deaminase, an aminotransferase, a hydratase, a superoxide dismutase, a ligase, a carbonic anhydrase, a nucleotide transferase, a glycosyltransferase, or a glycosidase.

In some embodiments, the POI is an ubiquitin ligase (i.e., a E3 ligase).

In some embodiments, the POI is a protein associated with a disease or disorder. In some embodiments, the protein does not have any known function in the mechanism of the disease or disorder.

In some embodiments, the first protein (e.g., the POI) is associated with a disease or disorder.

In some embodiments, the presence or activity of the first protein (e.g., the POI) is associated with a disease or disorder.

In some embodiments, certain post-translational modifications of the first protein are associated with a disease or disorder.

In some embodiments, the degradation of the first protein (e.g., the POI) results in treatment or prevention of the disease or disorder.

In some embodiments, the second protein is capable of causing the degradation of the first protein, under the induced proximity between the first protein and the second protein (e.g., in the cellular context or when other components, such as the proteasome machinery, are provided in the biochemical system).

In some embodiments, the second protein causes the degradation of the first protein, under the induced proximity between the first protein and the second protein (e.g., in the cellular context or when other components, such as the proteasome machinery, are provided in the biochemical system).

In some embodiments, the second protein is a ubiquitin ligase (i.e., a E3 ligase).

In some embodiments the fragment of the second protein comprises a binding site of the first protein.

In some embodiments the fragment of the second protein comprises at least a portion of the binding site of the second protein to the identified compound.

In some embodiments the fragment of the second protein comprises the binding site of the second protein to the identified compound.

In some embodiments the fragment of the second protein comprises the binding site of a ubiquitin ligase.

In some embodiments the fragment of the second protein comprises the binding site of an E3 ligase.

In some embodiments the binding site of the E3 ligase comprises a substrate receptor component. In some embodiments the substrate receptor component comprises, but is not limited to, cereblon (CRBN), CRBN/DDB1, DCAF15, DCAF15/DDB1, VHL, VHL/EloB/EloC.

In some embodiments, the binding site of the E3 ligase comprises an adaptor protein component. In some embodiments, the adaptor protein component comprises, but is not limited to DDB1.

In some embodiments, the second protein is a protein of interest (POI).

In some embodiments, the second protein is a kinase, a phosphatase, a glycosylase, a deglycosylase, a methylase, a demethylase, a sumoylase, a deubiquitinase, an acetyl transferase, a deacetylase, a fatty acyl transferase, a protease, an isomerase, or an arginase.

In some embodiments, the second protein is a protease, proteasome, a component of the proteasome complex, an autophage receptor, or a component of the autophage receptor complex.

In some embodiments, the second protein is a protein with scaffolding function to bring other proteins together into a complex.

In some embodiments, the second protein is a metabolic enzyme, a transcription factor, a cell surface receptor (e.g., a GPCR or a receptor tyrosine kinase), an ion channel, a membrane embedded or associated enzyme (e.g., an adenylyl cyclase), an RNA splicing enzyme, a transporter, a DNA helicase, or a DNA endonuclease.

In some embodiments, the second protein is a hydroxylase, a dehydrogenase, a reductase, an oxidase, an oxygenase, an oxidoreductase, a carboxylase, a decarboxylase, a lyase, an aldolase, a desaturase, a mutase, an epimerase, an isomerase, a racemase, an esterase, an amidase, a deaminase, an aminotransferase, a hydratase, a superoxide dismutase, a ligase, a carbonic anhydrase, a nucleotide transferase, a glycosyltransferase, or a glycosidase.

In some embodiments, the second protein is a heat shock protein.

In some embodiments, the second protein is associated with a disease or disorder.

In some embodiments, the presence or activity of the second protein is associated with a disease or disorder.

In some embodiments, a post-translational modification of the second protein is associated with a disease or disorder.

In some embodiments, the degradation of the second protein results in treatment or prevention of the disease or disorder.

In some embodiments, the first protein is a POI and the second protein is a ubiquitin ligase.

In some embodiments, the first protein is a ubiquitin ligase and the second protein is a POI.

In some embodiments, both the first protein and the second protein are the same protein (e.g., a POI or a ubiquitin ligase). For example, the first protein and the second protein may homodimerize in the presence of a compound of interest. In another example, the first protein and the second protein are each a kinase which may homodimerize in the presence of a compound of interest, which may lead to cross-phosphorylation and activation. In yet another example, the first protein and the second protein are each an E3 ligase which may homodimerize or heterodimerize in the presence of a compound of interest, which may lead to cross-ubiquitination and degradation.

### Characterizations of the Formed Complex

In some embodiments, the assay mixture results in a complex comprising the first protein or fragment thereof, the second protein or fragment thereof, and the compound.

In some embodiments, the first protein or fragment thereof, or the second protein or fragment thereof, is covalently attached to an affinity component.

In some embodiments, the first protein or fragment thereof, or the second protein or fragment thereof, together with the affinity component, form a fusion protein (e.g., glutathion-S-transferase (GST) or maltose binding protein (MBP)).

In some embodiments, the method further comprises:
(ii-a) contacting the complex with an immobilized affinity binder targeting the affinity component (or the fusion protein), thereby forming an immobilized complex.

In some embodiments, step (ii-a) comprises contacting the assay mixture with a plate coated with the affinity binder.

In some embodiments, step (ii-a) comprises contacting the assay mixture with beads coated with the affinity binder.

In some embodiments, the affinity component and the affinity binder are selected, but not limited, from the combinations described in Table 2 below.

TABLE 2

| Affinity Component | Affinity Binder |
|---|---|
| (or Affinity Binder) | (or Affinity Component) |
| Biotin | Streptavidin |
| Biotin | Avidin |
| Biotin | Neutravidin |
| Desthiobiotin | Streptavidin |
| Desthiobiotin | Avidin |
| Desthiobiotin | Neutravidin |
| FLAG Tag | FLAG Tag Antibody |
| HA Tag | HA Tag Antibody |
| Myc Tag | Myc Tag Antibody |
| Poly-Histidine Tag | Nickel (Ni) |
| BC2 Tag | BC2 Tag Antibody |

In some embodiments, the affinity component is biotin, desthiobiotin, or a derivative thereof.

In some embodiments, the affinity binder is streptavidin, avidin, neutravidin, or a derivative thereof.

In some embodiments, the affinity component is biotin or a derivative thereof, and the affinity binder is streptavidin or a derivative thereof.

In some embodiments, step (ii-a) further comprises incubating the mixture with the immobilized affinity binder for a time ranging from about 1 minute to about 90 minutes (e.g., from about 30 minutes to about 60 minutes).

In some embodiments, in step (i) the assay mixture further comprises a GFP booster and step (ii-a) comprises incubating the mixture with the immobilized affinity binder for a time ranging from 1 minute to 30 minutes.

In some embodiments, the method further comprises:
(ii-b) detecting and/or isolating the immobilized complex, thereby identifying the compound.

In some embodiments, step (ii-b) comprises detecting the immobilized complex, thereby identifying the compound.

In some embodiments, step (ii-b) comprises isolating the immobilized complex, thereby identifying the compound.

In some embodiments, step (ii-b) comprises detecting and isolating the immobilized complex, thereby identifying the compound.

In some embodiments, step (ii) or step (ii-b) comprises repeating step (i) one or more times, thereby identifying the compound, the first protein or fragment thereof, and/or the second protein or fragment thereof, in the complex.

In some embodiments, step (ii) or step (ii-b) comprises repeating step (i) one or more times, thereby identifying the compound in the complex.

In some embodiments, step (ii) or step (ii-b) comprises repeating step (i) one or more times, thereby identifying the first protein or fragment thereof, in the complex.

In some embodiments, step (ii) or step (ii-b) comprises repeating step (i) one or more times, thereby identifying the second protein or fragment thereof, in the complex.

In some embodiments, step (ii) or step (ii-b) comprises repeating step (i) one or more times, thereby identifying the compound, the first protein or fragment thereof, and the second protein or fragment thereof, in the complex.

In some embodiments, step (ii) or step (ii-b) comprises repeating step (i) with less different first proteins or fragments thereof, less different second protein or fragments thereof, and/or less different candidate compounds (e.g., as compared to the previous occurrence of step (i)).

In some embodiments, step (ii) or step (ii-b) comprises repeating step (i) with one first protein or fragment thereof, one second protein, and/or one candidate compound.

In some embodiments, individual first protein, second protein, and candidate compound in the assay mixture with generated or enhanced an assay signal are tested in such a way to uniquely and unambiguously identify the specific combination of first protein, second protein, and candidate compound that form the complex in the assay mixture. For example, individual combinations of first protein and second protein may be tested in each sample using the same pool of the plurality of candidate compounds to identify the combination of first protein and second protein. And the individual candidate compounds may then be tested using the identified specific combination of the first protein and the second protein. Alternatively, individual candidate compounds may be tested in each sample using the same plurality of first protein and plurality of second protein to identify the candidate compound. And the specific candidate compound can be tested in each sample using a unique combination of the first protein and the second protein. Other methods can also be used as long as a unique combination of the first protein, second protein, and the candidate compound can be unambiguously identified.

In some embodiments, step (ii-b) comprises characterizing the complex with mass spectrometry (MS).

In some embodiments, step (ii-b) comprises reducing, denaturizing, alkylating and/or digesting the immobilized complex to form a mixture of peptides, and characterizing the mixture of peptides with MS, thereby identifying the first protein or fragment thereof, and/or the second protein or fragment thereof, in the complex.

In some embodiments, the mixture of peptides further comprises the candidate compound, and the MS characterization further identifies the candidate compound in the complex.

In some embodiments, step (ii-b) comprises isolating the complex, and characterizing the complex with MS.

In some embodiments, step (ii-b) comprises isolating the complex, dissociating the first protein or fragment thereof, and/or the second protein or fragment thereof, from the complex, and characterizing the dissociated protein with MS, thereby identifying the first protein or fragment thereof, and/or the second protein or fragment thereof, in the complex.

In some embodiments, step (ii-b) comprises isolating the complex, dissociating the first protein or fragment thereof, and/or the second protein or fragment thereof, from the complex, and characterizing the dissociated protein with MS, thereby identifying the first protein or fragment thereof, in the complex.

In some embodiments, step (ii-b) comprises isolating the complex, dissociating the first protein or fragment thereof, and/or the second protein or fragment thereof, from the complex, and characterizing the dissociated protein with MS, thereby identifying the second protein or fragment thereof, in the complex.

In some embodiments, step (ii-b) comprises isolating the complex, dissociating the first protein or fragment thereof, and/or the second protein or fragment thereof, from the complex, and characterizing the dissociated protein with MS, thereby identifying the first protein or fragment thereof, and the second protein or fragment thereof, in the complex.

In some embodiments, step (ii-b) further comprises dissociating the candidate compound from the complex, and the MS characterization further identifies the candidate compound in the complex.

In some embodiments, the MS characterization is based on a “bottom-up mass spectrometry-based proteomics” method. In such method, the proteins captured in the incubation may be reduced under denaturing condition and alkylated followed by digestion with a sequence-specific protease into smaller peptides before analysis of the samples by mass spectrometer. Various options may be available for this procedure. For example, a high concentration of urea or other chaotropic agents may be used for the denaturation of proteins. Dithiothreitol, beta-mercaptoethanol, or TCEP (tris(2-carboxyethyl)phosphine) may be used for reducing the disulfide bonds in the protein. Iodoacetamide, iodoacetic acid, or iodoethanol may be used as alkylating agents that covalently modify the free sulfhydryl group on the protein once the disulfide bonds are reduced. Trypsin may be used as a sequence-specific protease to digest proteins into peptides for the purpose of mass spectrometry-based protein analysis. Other proteases that may be used for this purpose include Lys-C and chymotrypsin. Amino acid sequences of the digested peptides may then be identified by the standard proteomics method. Such method may be based on comparing the acquired m/z (mass to charge ratio) value of the peptides and their MS2 fragmentation spectra with the theoretical data obtained from in-silico digestion of the protein sequences in the protein database using a computer program (also known as “search engine”) for the appropriate species. For mass spectrometry data acquisition, in-line separation of the peptides on liquid chromatography (LC) may be combined with different modes of data acquisition by the mass spectrometer. Data-dependent MS2 acquisition (DDA) and/or Data-independent acquisition (DIA) may be suitable for such purpose. Any other data acquisition methods may also be used as long as unambiguous identification of the unique peptide sequences can be established. Direct acquisition of data may be done without LC separation of the peptides especially when the sample composition is relatively simple. Matrix-assisted laser desorption/ionization (MALDI) time-of-flight instrument may be suitable for such purpose. Identity of the protein(s) in the sample may be deduced from comparison of the amino acid sequence of the peptides with those of the intact proteins in the protein database. During digestion of the proteins in the complex, the candidate compound trapped in the complex may also be released into the digestion solution. The identity of the candidate compound may also be obtained from the same sample, and/or from the same experiment, by comparing the molecular weight of the compounds in the assay mixture with the expected molecular weight of the candidate compound deduced from the m/z values of the MS1 spectra. When necessary, MS2 fragmentation spectra of the compound may also be used to aid identification of the compound.

In some embodiments, the MS characterization is based on a “top-down” method (e.g., without digestion of the protein if the proteins in the complex can be released into the solution). In this method, the intact protein mass obtained by the mass spectrometer may be compared to the expected mass of the individual proteins used in this experiment. To release the intact protein from the complex, one may introduce a short peptide sequence between the first protein or the second protein and the first or second tag fragment (e.g., the GFP10 or GFP11 tag fragment), which may be cleaved by a highly sequence specific protease. For example, the short peptide sequence may be a TEV cleavage sequence, ENLYFQS(G,A). The TEV protease could cleave between the Q and S in a highly sequence specific manner. Thus, the entire protein may be released from the complex bound to the plate, e.g., by incubating the plate with a solution containing the TEV protease. Once the first or second protein is severed from the tag fragment, acidification of the sample can dissociate the two proteins from the ternary complex. Measurement of the intact mass of the dissociated proteins can lead to identification of specific proteins within the ternary complex.

In some embodiments, the method further comprises:
(ii-c) subjecting the identified compound to a verifying assay.

In some embodiments, the verifying assay is configured to test the capability of a compound toward inducing proximity between the first protein or fragment thereof, and the second protein or fragment thereof.

In some embodiments, the verifying assay is configured to test the capability of a compound toward inducing degradation of the first protein (e.g., the POI), in the presence of the second protein (e.g., the E3 ligase).

In some embodiments, in step (ii-c), the identified compound results in an induced proximity between the first protein or fragment thereof, and the second protein or fragment thereof, in the verifying assay.

In some embodiments, in step (ii-c), the identified compound results in a degradation of the first protein (e.g., the POI), in the presence of the second protein (e.g., the E3 ligase), in the verifying assay.

### Identified First Proteins, Second Proteins, Compounds, and Combinations

In some aspects, the present disclosure provides a first protein identified by the method of the present disclosure.

In some aspects, the present disclosure provides a second protein identified by the method of the present disclosure.

In some aspects, the present disclosure provides a combination of a first protein and a second protein, identified by the method of the present disclosure.

In some aspects, the present disclosure provides a combination of a first protein, a second protein, and a compound targeting the first protein and the second protein, identified by the method of the present disclosure.

In some aspects, the present disclosure provides a compound identified by the method of the present disclosure.

In some embodiments, the identified compound is a binder compound.

In some embodiments, the identified compound (e.g., binder compound) is capable of modulating a protein-protein interaction (PPI) between the first protein or fragment thereof, and the second protein or fragment thereof.

In some embodiments, the identified compound (e.g., binder compound) modulates (increases or decreases) a protein-protein interaction (PPI) between the first protein and the second protein.

In some embodiments, the identified compound (e.g., binder compound) increases protein-protein interaction (PPI) between the first protein and the second protein or stabilizes the PPI between the two proteins.

In some embodiments, the identified compound (e.g., binder compound) decreases protein-protein interaction (PPI) between the first and the second protein or destabilizes the PPI between the two proteins.

In some embodiments, the PPI results in the degradation of the first protein or the second protein.

In some embodiments, the PPI results in increased stability of the protein within the cells or protection against proteolysis.

In some embodiments, the PPI results in a post-translational modification of the first protein or the second protein.

In some embodiments, the PPI results in removal of pre-existing post-translational modification of the first protein or the second protein

In some embodiments, the PPI results in changes in subcellular localization of the first protein or the second protein

In some embodiments, wherein the PPI results in a modulation of the activity of the first protein or the second protein.

In some embodiments, the identified compound (e.g., binder compound) is capable of causing the degradation of the first protein (e.g., the POI), in the presence of the second protein (e.g., the E3 ligase).

In some embodiments, the identified compound (e.g., binder compound) causes the degradation of the first protein (e.g., the POI), in the presence of the second protein (e.g., the E3 ligase).

In some embodiments, the identified compound (e.g., binder compound) is capable of causing the post-translational modification of the first protein (e.g., the POI), in the presence of the second protein (e.g., the E3 ligase).

In some embodiments, the identified compound (e.g., binder compound) causes the post-translational modification of the first protein (e.g., the POI), in the presence of the second protein (e.g., the E3 ligase).

In some embodiments, the identified compound (e.g., binder compound) is capable of causing the modulation of the activity of the first protein (e.g., the POI), in the presence of the second protein (e.g., the E3 ligase).

In some embodiments, the identified compound (e.g., binder compound) causes the modulation of the activity of the first protein (e.g., the POI), in the presence of the second protein (e.g., the E3 ligase).

### Methods of Synthesizing the Identified Compounds

In some aspects, the present disclosure provides a method of preparing a compound identified in a method of the present disclosure.

The compounds of the present disclosure can be prepared by any suitable technique known in the art. It is understood by one skilled in the art of organic synthesis that the functionality present on various portions of the molecule must be compatible with the compounds and reaction conditions utilized. The resultant compounds can be isolated and purified using techniques well known in the art.

Conveniently, the reaction of the compounds is carried out in the presence of a suitable solvent, which is preferably inert under the respective reaction conditions. Examples of suitable solvents comprise but are not limited to hydrocarbons, such as hexane, petroleum ether, benzene, toluene or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF), 2-methyltetrahydrofuran, cyclopentylmethyl ether (CPME), methyl tert-butyl ether (MTBE) or dioxane; glycol ethers, such as ethylene glycol monomethyl or monoethyl ether or ethylene glycol dimethyl ether (diglyme); ketones, such as acetone, methylisobutylketone (MIBK) or butanone; amides, such as acetamide, dimethylacetamide, dimethylformamide (DMF) or N-methylpyrrolidinone (NMP); nitriles, such as acetonitrile; sulfoxides, such as dimethyl sulfoxide (DMSO); nitro compounds, such as nitromethane or nitrobenzene; esters, such as ethyl acetate or methyl acetate, or mixtures of the said solvents or mixtures with water.

As will be understood by the person skilled in the art of organic synthesis, the compounds are readily accessible by various synthetic routes, some of which are exemplified in the accompanying examples. The skilled person will easily recognize which kind of compounds and reactions conditions are to be used and how they are to be applied and adapted in any particular instance—wherever necessary or useful—in order to obtain the compounds of the present disclosure. Furthermore, some of the compounds can readily be synthesized by reacting other compounds of the present disclosure under suitable conditions, for instance, by converting one particular functional group present in a compound of the present disclosure, or a suitable precursor molecule thereof, into another one by applying standard synthetic methods, like reduction, oxidation, addition or substitution reactions; those methods are well known to the skilled person. Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P. G. M. Wuts, T. W. Greene, “Greene's Protective Groups in Organic Synthesis”, 4th edition (2006) (John Wiley & Sons).

### Verifying Assays for the Identified Compounds

Compounds identified by methods described above, once produced, can be verified using a variety of assays known to those skilled in the art to determine whether the compounds have biological activity (e.g., to induce proximity between the first protein or fragment thereof, and the second protein or fragment thereof). For example, the compounds can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds. These in vitro or in vivo biological assays can include, but are not limited to, enzymatic activity assays, electrophoretic mobility shift assays, reporter gene assays, in vitro cell viability assays, and the assays described herein.

In some embodiments, the verifying assay are biological assays that verify the formation of a ternary complex. In some embodiments, the verifying assay measures the proximity between the first protein and the second protein, or a fragment thereof, in a compound dependent manner. In some embodiments, the verifying assay can include, but is not limited to, AlphALISA, TR-FRET, Fluorescence polarization assay, Surface Plasmon Resonance (SPR), mass photometry. In some embodiments, the verifying assay comprises pull-down of the ternary complex and quantification of the same by mass spectrometry.

In some embodiments, the verifying assay is an intracellular biological assay. In some embodiments, the intracellular biological assay comprises a nanoBRET system. In some embodiments, the nanoBRET system measures the formation of the ternary complex inside the cell. In some embodiments, the nanoBRET system further comprises detection of the generation or enhancement of a luminescence signal upon formation the ternary complex comprising interaction of the first protein and second protein, or a fragment thereof, in the presence of the compound. In some embodiments, the intracellular biological assay comprises a fluorescence resonance energy transfer (FRET) system. In some embodiments, the FRET system measures the formation of the ternary complex inside the cell. In some embodiments, the FRET system further comprises detection of the generation or enhancement of a signal upon formation the ternary complex comprising interaction of the first protein and second protein, or a fragment thereof, in the presence of the compound, wherein the first protein and second protein, or a fragment thereof, are fused with a fluorescence donor and/or fluorescence acceptor.

In some embodiments, the verifying assay is a cell-based degradation assay. In some embodiments, the cell-based degradation assay can include, but is not limited to, western blots, in cell western assays, HiBit assays, fluorescence reporter assay, and mass spectrometry proteomics.

Without wishing to be bound by theory, compound-dependent ubiquitination, post-translational modification, change in enzymatic activity or cellular degradation of the POI in a cell-based degradation verifying assay does not directly measure the formation of the ternary complex comprising the first protein and second protein, or a fragment thereof, in the presence of the compound, but requires the formation of the ternary complex, and therefore can be used to detect ternary complex formation.

### Pharmaceutical Compositions

In some aspects, the present disclosure provides a pharmaceutical composition comprising a compound identified by a method of the present disclosure, or a pharmaceutically acceptable salt or solvate thereof, and one or more pharmaceutically acceptable carriers or excipients.

As used herein, the term “composition” is intended to encompass a product comprising the specified ingredients in the specified amounts, as well as any product which results, directly or indirectly, from combination of the specified ingredients in the specified amounts. The compounds of present disclosure can be formulated for oral administration in forms such as tablets, capsules (each of which includes sustained release or timed-release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

Any suitable solubility enhancing compound can be used. Examples of a solubility enhancing compound include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulfated β-cyclodextrin (S-3-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating compound can be used. Examples of a suitable chelating compound include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity compound to adjust the tonicity (osmotic pressure). The tonicity compound can be selected from the group consisting of a glycol (such as propylene glycol, diethylene glycol, triethylene glycol), glycerol, dextrose, glycerin, mannitol, potassium chloride, and sodium chloride, and a mixture thereof. The aqueous vehicle may also contain a viscosity/suspending compound. Suitable viscosity/suspending compounds include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof. The aqueous vehicle may also contain a buffering compound to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and F-aminocaproic acid, and mixtures thereof.

The composition may further comprise a wetting compound. Suitable classes of wetting compounds include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

The compositions of the disclosure may be obtained by conventional procedures using conventional pharmaceutical excipients, well known in the art. Thus, compositions intended for oral use may contain, for example, one or more coloring, sweetening, flavoring and/or preservative compounds.

### Methods of Using the Identified Compounds

In some aspects, the present disclosure provides a method of degrading a first protein in a subject, comprising administering to the subject a compound identified by the method of the present disclosure.

In some aspects, the present disclosure provides a compound identified by the method of the present disclosure, for use in degrading a first protein in a subject.

In some aspects, the present disclosure provides a use of a compound identified by the present disclosure in the manufacture of a medicament for degrading a first protein in a subject.

In some aspects, the present disclosure provides a method of treating and/or preventing a disease or disorder associated with a first protein in a subject, comprising administering to the subject a therapeutically effective amount of a compound identified by the method of the present disclosure.

In some aspects, the present disclosure provides a compound identified by the method of the present disclosure for use in treating and/or preventing a disease or disorder associated with a first protein in a subject.

In some aspects, the present disclosure provides a use of a compound identified by the method of the present disclosure in the manufacture of a medicament for treating and/or preventing a disease or disorder associated with a first protein in a subject.

In some embodiments, the disease or disorder is associated with the first protein (e.g., the POI).

In some embodiments, the disease or disorder is mediated by the first protein (e.g., the POI).

In some aspects, the present disclosure provides a method of degrading a second protein in a subject, comprising administering to the subject a compound identified by the method of the present disclosure.

In some aspects, the present disclosure provides a compound identified by the method of the present disclosure, for use in degrading a second protein in a subject.

In some aspects, the present disclosure provides a use of a compound identified by the present disclosure in the manufacture of a medicament for degrading a second protein in a subject.

In some aspects, the present disclosure provides a method of treating and/or preventing a disease or disorder associated with a second protein in a subject, comprising administering to the subject a therapeutically effective amount of a compound identified by the method of the present disclosure.

In some aspects, the present disclosure provides a compound identified by the method of the present disclosure for use in treating and/or preventing a disease or disorder associated with a second protein in a subject.

In some aspects, the present disclosure provides a use of a compound identified by the method of the present disclosure in the manufacture of a medicament for treating and/or preventing a disease or disorder associated with a second protein in a subject.

In some embodiments, the disease or disorder is associated with the second protein (e.g., the E3 ligase).

In some embodiments, the disease or disorder is mediated by the second protein.

In some embodiments, the subject is a cell.

In some embodiments, the subject is a mammal.

In some embodiments, the subject is a human.

In some embodiments, the disease or disorder is cancer.

In some embodiments, the cancer is selected from prostate cancer (small cell carcinomas, neuroendocrine tumors, transitional cell carcinomas, sarcomas), breast cancer (ductal carcinoma in situ, invasive breast cancer, triple-negative breast cancer (TNBC), inflammatory breast cancer, Paget disease of the breast, Angiosarcoma, Phyllodes tumor), ovarian cancer (epithelial ovarian carcinomas, germ cell tumors, stromal cell tumors), bladder cancer (urothelial carcinoma, squamous cell carcinoma, adenocarcinoma), stomach cancer (adenocarcinoma, primary gastric lymphoma, gastrointestinal stromal tumor, and neuroendocrine carcinoid tumors), pancreatic cancer (adenocarcinoma and neuroendocrine tumors), liver cancer (hepatocellular carcinoma, cholangiocarcinoma), endometrial cancer, salivary gland carcinoma, leukemias, NUT-midline carcinoma, multiple myeloma, lung cancer (small cell lung cancer, non small cell lung cancer), neuroblastoma, cervical cancer (squamous cell cancer, adenocarcinoma), esophageal cancer, colorectal cancer, brain (gliomas), glioblastomas, Bannayan-Zonana syndrome, Cowden disease, Lhermitte-Duclos disease, Wilm's tumor, Ewing's sarcoma, Rhabdomyosarcoma, ependymoma, medulloblastoma, colon cancer (primary colorectal lymphomas, gastrointestinal stromal tumors, Leiomyosarcomas, carcinoid tumors, and melanomas), head and neck cancer, lung cancer (adenocarcinoma, squamous cell carcinoma, and large cell carcinoma), skin cancer (basal cell carcinoma, squamous cell carcinoma), bone marrow cancer (melanoma, lymphoma, myeloma), renal cancer (renal cell carcinoma, urothelial carcinoma, Wilms tumor), sarcoma, bone cancer (osteosarcoma, Ewing's sarcoma, chondrosarcoma, fibrosarcoma, giant cell tumor of bone, chordoma, multiple myeloma), and thyroid cancer.

In some embodiments, the disease or disorder is a benign proliferative disorder.

In some embodiments, the benign proliferative disorder is selected from benign soft tissue tumors, bone tumors, brain and spinal tumors, eyelid and orbital tumors, granuloma, lipoma, meningioma, multiple endocrine neoplasia, nasal polyps, pituitary tumors, prolactinoma, pseudotumor cerebri, seborrheic keratoses, stomach polyps, thyroid nodules, cystic neoplasms of the pancreas, hemangiomas, vocal cord nodules, polyps, and cysts, Castleman disease, chronic pilonidal disease, dermatofibroma, pilar cyst, pyogenic granuloma, and juvenile polyposis syndrome.

In some embodiments, the disease or disorder is an immunological disease or disorder.

In some embodiments, the immunological disease or disorder comprises T-cell mediated inflammatory diseases and B-cell mediated inflammatory diseases. In some embodiments, the immunological disease or disorder is selected from Crohn's disease, ulcerative colitis, Lupus, cystic fibrosis, childhood asthma, adult asthma, allergic disease, chronic obstructive pulmonary disease, psoriasis, atherosclerosis, acute and chronic inflammation, Addison disease, celiac disease—sprue (gluten-sensitive enteropathy), dermatomyositis, Graves disease, Hashimoto thyroiditis, multiple sclerosis, myasthenia gravis, pernicious anemia, reactive arthritis, rheumatoid arthritis, Sjögren syndrome, systemic lupus erythematosus, Type I diabetes, inflammatory bowel disease, chronic inflammatory demyelinating polyneuropathy, and ankylosing spondylitis.

In some embodiments, the disease or disorder is a neurological disease or disorder.

In some embodiments, the neurological disease or disorder is selected from a list that includes, but is not limited to, Acute Spinal Cord Injury, Alzheimer's Disease, Amyotrophic Lateral Sclerosis (ALS), Ataxia, Bell's Palsy, Brain Tumors, Cerebral Aneurysm, Dementia, Epilepsy and Seizures, Guillain-Barré Syndrome, Huntington's Disease, Headache, Head Injury, Hydrocephalus, Lumbar Disk Disease (Herniated Disk), Meningitis, Multiple Sclerosis, Muscular Dystrophy, Neurocutaneous Syndromes, Parkinson's Disease, Stroke (Brain Attack), Cluster Headaches, Tension Headaches, Migraine Headaches, Encephalitis, Septicemia, Myasthenia Gravis, Muscular Dystrophy and Neuromuscular Diseases.

### EXEMPLARY EMBODIMENTS

Exemplary Embodiment No. 1. A method of identifying a first protein, a second protein, a compound targeting the first protein and the second protein, or any combination thereof, comprising:
(i) providing an assay mixture comprising:
   (a) a first protein or a fragment thereof, covalently attached to a first tag fragment;
   (b) a second protein or a fragment thereof, covalently attached to a second tag fragment, wherein the second tag fragment is complementary to the first tag fragment; and
   (c) a candidate compound,
   wherein the first tag fragment and the second tag fragment are configured to generate or enhance an assay signal upon the assay mixture resulting in a complex comprising the first protein or fragment thereof, the second protein or fragment thereof, and the compound; and
(ii) identifying the first protein, the second protein, the compound, or any combination thereof associated with the complex.

Exemplary Embodiment No. 2. A method of identifying a first protein, a second protein, a compound targeting the first protein and the second protein, or any combination thereof, comprising:
(i) providing an assay mixture comprising:
   (a) a first protein or a fragment thereof, covalently attached to a first tag fragment;
   (b) a second protein or a fragment thereof, covalently attached to a second tag fragment, wherein the second tag fragment is complementary to the first tag fragment; and
   (c) a candidate compound,
   wherein the first protein or fragment thereof, and/or the second protein or fragment thereof, is covalently attached to an affinity component; and
   wherein the first tag fragment and the second tag fragment are configured to generate or enhance an assay signal upon the assay mixture resulting in a complex comprising the first protein or fragment thereof, the second protein or fragment thereof, and the compound;
(ii-a) contacting the complex with an immobilized affinity binder targeting the affinity component, thereby forming an immobilized complex; and
(ii-b) detecting and/or isolating the immobilized complex, thereby identifying the first protein, the second protein, the compound, or any combination thereof.

Exemplary Embodiment No. 3. A method of identifying a first protein, a second protein, a compound targeting the first protein and the second protein, or any combination thereof, comprising:
(i) providing an assay mixture comprising:
   (a) a first protein or a fragment thereof, covalently attached to a first tag fragment;
   (b) a second protein or a fragment thereof, covalently attached to a second tag fragment, wherein the second tag fragment is complementary to the first tag fragment; and
   (c) a candidate compound,
   wherein the first tag fragment and the second tag fragment are configured to generate or enhance an assay signal upon the assay mixture resulting in an induced proximity between the first protein or fragment thereof, and the second protein or fragment thereof; and
(ii) identifying the first protein, the second protein, the compound, or any combination thereof associated with the induced proximity;
   wherein the identified compound is capable of causing the degradation of the first protein (e.g., the POI), in the presence of the second protein.

Exemplary Embodiment No. 4. The method of any one of the preceding Exemplary Embodiments, wherein the assay mixture results in induced proximity such that the assay signal is generated or enhanced.

Exemplary Embodiment No. 5. The method of any one of the preceding Exemplary Embodiments, wherein the assay mixture further comprises a detector fragment complementary to the first tag fragment and the second tag fragment.

Exemplary Embodiment No. 6. The method of any one of the preceding Exemplary Embodiments, wherein the first tag fragment, the second tag fragment, and the detector fragment are configured to generate or enhance an assay signal upon the assay mixture resulting in an induced proximity between the first protein or fragment thereof, and the second protein or fragment thereof.

Exemplary Embodiment No. 7. The method of any one of the preceding Exemplary Embodiments, wherein the first tag fragment, the second tag fragment, and the detector fragment are configured to generate or enhance an assay signal upon the assay mixture resulting in a complex comprising the first protein or fragment thereof, the second protein or fragment thereof, and the compound.

Exemplary Embodiment No. 8. The method of any one of the preceding Exemplary Embodiments, wherein the assay mixture comprises a plurality of different first proteins or fragments thereof.

Exemplary Embodiment No. 9. The method of any one of the preceding Exemplary Embodiments, wherein the assay mixture comprises a plurality of different second proteins or fragments thereof.

Exemplary Embodiment No. 10. The method of any one of the preceding Exemplary Embodiments, wherein the assay mixture comprises a plurality of different candidate compounds.

Exemplary Embodiment No. 11. The method of any one of the preceding Exemplary Embodiments, wherein the assay mixture comprises from about 50 to about 250 different candidate compounds.

Exemplary Embodiment No. 12. The method of any one of the preceding Exemplary Embodiments, wherein the assay mixture comprises from about 250 to about 2500 different candidate compounds.

Exemplary Embodiment No. 13. The method of any one of the preceding Exemplary Embodiments, wherein the assay mixture comprises more than about 2500 different candidate compounds.

Exemplary Embodiment No. 14. The method of any one of the preceding Exemplary Embodiments, wherein the assay mixture results in the complex comprising the first protein or fragment thereof, the second protein or fragment thereof, and the candidate compound.

Exemplary Embodiment No. 15. The method of any one of the preceding Exemplary Embodiments, wherein the assay signal is fluorescence.

Exemplary Embodiment No. 16. The method of any one of the preceding Exemplary Embodiments, wherein the first tag fragment is a first tag GFP fragment.

Exemplary Embodiment No. 17. The method of any one of the preceding Exemplary Embodiments, wherein the second tag fragment is a second tag GFP fragment.

Exemplary Embodiment No. 18. The method of any one of the preceding Exemplary Embodiments, wherein the detector fragment is a detector GFP fragment.

Exemplary Embodiment No. 19. The method of any one of the preceding Exemplary Embodiments, wherein the first tag GFP fragment, the second tag GFP fragment, and the detector GFP fragment are configured to form a GFP upon the assay mixture resulting in an induced proximity between the first protein or fragment thereof, and the second protein or fragment thereof.

Exemplary Embodiment No. 20. The method of any one of the preceding Exemplary Embodiments, wherein the first tag GFP fragment and the second tag GFP fragment independently comprises one or more of GFP1, GFP2, GFP3, GFP4, GFP5, GFP6, GFP7, GFP8, GFP9, GFP10, and GFP11.

Exemplary Embodiment No. 21. The method of any one of the preceding Exemplary Embodiments, wherein the first tag GFP fragment and the second tag GFP fragment independently comprises one or more of GFP10 and GFP11.

Exemplary Embodiment No. 22. The method of any one of the preceding Exemplary Embodiments, wherein the first tag GFP fragment comprises GFP10, and the second tag GFP fragment comprises GFP11.

Exemplary Embodiment No. 23. The method of any one of the preceding Exemplary Embodiments, wherein the detector GFP fragment is selected from GFP1, GFP2, GFP3, GFP4, GFP5, GFP6, GFP7, GFP8, and GFP9.

Exemplary Embodiment No. 24. The method of any one of the preceding Exemplary Embodiments, wherein the first protein is a protein of interest (POI).

Exemplary Embodiment No. 25. The method of any one of the preceding Exemplary Embodiments, wherein the second protein is a ubiquitin ligase.

Exemplary Embodiment No. 26. The method of any one of the preceding Exemplary Embodiments, wherein step (i) further comprises detecting the generated or enhanced assay signal.

Exemplary Embodiment No. 27. The method of any one of the preceding Exemplary Embodiments, wherein the assay signal is fluorescence.

Exemplary Embodiment No. 28. The method of any one of the preceding Exemplary Embodiments, wherein the first protein or fragment thereof, or the second protein or fragment thereof, is covalently attached to an affinity component.

Exemplary Embodiment No. 29. The method of any one of the preceding Exemplary Embodiments, wherein the affinity component is biotin.

Exemplary Embodiment No. 30. The method of any one of the preceding Exemplary Embodiments, wherein the method further comprises:
(ii-a) contacting the complex with an immobilized affinity binder targeting the affinity component, thereby forming an immobilized complex.

Exemplary Embodiment No. 31. The method of any one of the preceding Exemplary Embodiments, wherein step (ii-a) comprises contacting the assay mixture with a plate coated with the affinity binder.

Exemplary Embodiment No. 32. The method of any one of the preceding Exemplary Embodiments, wherein the affinity binder is streptavidin.

Exemplary Embodiment No. 33. The method of any one of the preceding Exemplary Embodiments, wherein step (ii-a) further comprises incubating the assay mixture with the plate for a time ranging from 1 minute to 90 minutes.

Exemplary Embodiment No. 34. The method of any one of the preceding Exemplary Embodiments, wherein in step (i) the assay mixture further comprises a GFP booster, and wherein step (ii-a) comprises incubating the assay mixture with the plate for a time ranging from 1 minute to 30 minutes.

Exemplary Embodiment No. 35. The method of any one of the preceding Exemplary Embodiments, wherein the method further comprises:
(ii-b) detecting and/or isolating the immobilized complex, thereby identifying the compound.

Exemplary Embodiment No. 36. The method of any one of the preceding Exemplary Embodiments, wherein step (ii) or step (ii-b) comprises repeating step (i) one or more times with less different first proteins or fragments thereof, less different second protein or fragments thereof, and/or less different candidate compounds, as compared to the previous occurrence of step (i), thereby identifying the compound, the first protein or fragment thereof, and/or the second protein or fragment thereof, in the complex.

Exemplary Embodiment No. 37. The method of any one of the preceding Exemplary Embodiments, wherein step (ii) or step (ii-b) comprises repeating step (i) one or more times with less different first proteins or fragments thereof, less different second protein or fragments thereof, and/or less different candidate compounds, as compared to the previous occurrence of step (i), thereby identifying the compound in the complex.

Exemplary Embodiment No. 38. The method of any one of the preceding Exemplary Embodiments, wherein step (ii) or step (ii-b) comprises repeating step (i) one or more times with less different first proteins or fragments thereof, less different second protein or fragments thereof, and/or less different candidate compounds, as compared to the previous occurrence of step (i), thereby the first protein or fragment thereof, in the complex.

Exemplary Embodiment No. 39. The method of any one of the preceding Exemplary Embodiments, wherein step (ii) or step (ii-b) comprises repeating step (i) one or more times with less different first proteins or fragments thereof, less different second protein or fragments thereof, and/or less different candidate compounds, as compared to the previous occurrence of step (i), thereby identifying the second protein or fragment thereof, in the complex.

Exemplary Embodiment No. 40. The method of any one of the preceding Exemplary Embodiments, wherein step (ii-b) comprises characterizing the complex with mass spectrometry (MS).

Exemplary Embodiment No. 41. The method of any one of the preceding Exemplary Embodiments, wherein step (ii-b) comprises reducing, denaturizing, alkylating and/or digesting the immobilized complex to form a mixture of peptides and characterizing the mixture of peptides with MS, thereby identifying the first protein or fragment thereof, and/or the second protein or fragment thereof, in the complex.

Exemplary Embodiment No. 42. The method of any one of the preceding Exemplary Embodiments, wherein step (ii-b) comprises reducing, denaturizing, alkylating and/or digesting the immobilized complex to form a mixture of peptides and characterizing the mixture of peptides with MS, thereby identifying the first protein or fragment thereof, in the complex.

Exemplary Embodiment No. 43. The method of any one of the preceding Exemplary Embodiments, wherein step (ii-b) comprises reducing, denaturizing, alkylating and/or digesting the immobilized complex to form a mixture of peptides and characterizing the mixture of peptides with MS, thereby identifying the second protein or fragment thereof, in the complex.

Exemplary Embodiment No. 44. The method of any one of the preceding Exemplary Embodiments, wherein step (ii-b) comprises reducing, denaturizing, alkylating and/or digesting the immobilized complex to form a mixture of peptides and characterizing the mixture of peptides with MS, thereby identifying the first protein or fragment thereof, and the second protein or fragment thereof, in the complex.

Exemplary Embodiment No. 45. The method of any one of the preceding Exemplary Embodiments, wherein the mixture of peptides further comprises the compound, and the MS characterization further identifies the compound in the complex.

Exemplary Embodiment No. 46. The method of any one of the preceding Exemplary Embodiments, wherein step (ii-b) comprises isolating the complex, dissociating the first protein or fragment thereof, and/or the second protein or fragment thereof, from the complex, and characterizing the dissociated protein with MS, thereby identifying the first protein or fragment thereof, and/or the second protein or fragment thereof, in the complex.

Exemplary Embodiment No. 47. The method of any one of the preceding Exemplary Embodiments, wherein step (ii-b) further comprises dissociating the candidate compound from the immobilized complex, and the MS characterization further identifies the candidate compound in the complex.

Exemplary Embodiment No. 48. The method of any one of the preceding Exemplary Embodiments, wherein the identified compound modulates a protein-protein interaction (PPI) between the first protein and the second protein.

Exemplary Embodiment No. 49. The method of any one of the preceding Exemplary Embodiments, wherein the PPI results in the degradation of the first protein or the second protein.

Exemplary Embodiment No. 50. The method of any one of the preceding Exemplary Embodiments, wherein the PPI results in the stabilization of the first protein or the second protein.

Exemplary Embodiment No. 51. The method of any one of the preceding Exemplary Embodiments, wherein the identified compound causes a degradation of the first protein in the presence of the second protein or degradation of the second protein in the presence of the first protein.

Exemplary Embodiment No. 52. The method of any one of the preceding Exemplary Embodiments, wherein the PPI results in the post-translational modification of the first protein or the second protein.

Exemplary Embodiment No. 53. The method of any one of the preceding Exemplary Embodiments, wherein the PPI results in removal of the pre-existing post-translational modification of the first protein or the second protein.

Exemplary Embodiment No. 54. The method of any one of the preceding Exemplary Embodiments, wherein the PPI results in the modulation of the activity of the first protein or the second protein.

Exemplary Embodiment No. 55. The method of any one of the preceding Exemplary Embodiments, wherein the PPI results in the changes in subcellular localization of the first protein or the second protein.

Exemplary Embodiment No. 56. The method of any one of the preceding Exemplary Embodiments, wherein the second protein is associated with a disease or disorder.

Exemplary Embodiment No. 57. The method of any one of the preceding Exemplary Embodiments, wherein the method is performed one or more times.

Exemplary Embodiment No. 58. The method of any one of the preceding Exemplary Embodiments, wherein the method is performed using new wells each time.

Exemplary Embodiment No. 59. A first protein identified by the method of any one of the preceding Exemplary Embodiments.

Exemplary Embodiment No. 60. A second protein identified by the method of any one of the preceding Exemplary Embodiments.

Exemplary Embodiment No. 61. A combination of a first protein and a second protein, identified by the method of any one of the preceding Exemplary Embodiments.

Exemplary Embodiment No. 62. A combination of a first protein, a second protein, and a compound targeting the first protein and the second protein, identified by the method of any one of the preceding Exemplary Embodiments.

Exemplary Embodiment No. 63. A compound identified by the method of any one of the preceding Exemplary Embodiments.

Exemplary Embodiment No. 64. A pharmaceutical composition comprising a compound identified by the method of any one of the preceding Exemplary Embodiments.

Exemplary Embodiment No. 65. A method of degrading a first protein or a second protein in a subject, comprising administering to the subject a compound identified by the method of any one of the preceding Exemplary Embodiments.

Exemplary Embodiment No. 66. A method of modulating a PPI between a first protein and a second protein in a subject, comprising administering to the subject a compound identified by the method of any one of the preceding Exemplary Embodiments.

Exemplary Embodiment No. 67. A compound identified by the method of any one of the preceding Exemplary Embodiments, for use in degrading a first protein or a second protein in a subject.

Exemplary Embodiment No. 68. A compound identified by the method of any one of the preceding Exemplary Embodiments, for use in modulating a PPI between a first protein and a second protein in a subject.

Exemplary Embodiment No. 69. Use of a compound identified by the method of any one of the preceding Exemplary Embodiments in the manufacture of a medicament for degrading a first protein or a second protein in a subject.

Exemplary Embodiment No. 70. Use of a compound identified by the method of any one of the preceding Exemplary Embodiments in the manufacture of a medicament for modulating a PPI between a first protein and a second protein in a subject.

Exemplary Embodiment No. 71. A method of treating and/or preventing a disease or disorder associated with a first protein or a second protein in a subject, comprising administering to the subject a therapeutically effective amount of a compound identified by the method of any one of the preceding Exemplary Embodiments.

Exemplary Embodiment No. 72. A compound identified by the method of any one of the preceding Exemplary Embodiments for use in treating and/or preventing a disease or disorder associated with a first protein or a second protein in a subject.

Exemplary Embodiment No. 73. Use of a compound identified by the method of any one of the preceding Exemplary Embodiments in the manufacture of a medicament for treating and/or preventing a disease or disorder associated with a first protein or a second protein in a subject.

### Definitions

Unless otherwise stated, the following terms used in the specification and claims have the following meanings set out below.

It is understood that, unless indicated otherwise, when referring to the amount of the first protein or fragment thereof, the second protein or fragment thereof, and the candidate compound in the assay mixture, the present disclosure intends to describe the number of different structures, instead of molecular amount, of the first protein or fragment thereof, the second protein or fragment thereof, and the candidate compound. For example, when the assay mixture comprises “a first protein”, then the first protein(s) present in the assay mixture may have the same structure or different structures. When the assay mixture comprises “a plurality of first proteins”, then the first protein(s) present in the assay mixture have at least two different structures. For another example, when the assay mixture comprises “a second protein”, then the second protein(s) present in the assay mixture may have the same structure or different structures. When the assay mixture comprises “a plurality of second proteins”, then the second protein(s) present in the assay mixture have at least two different structures. For another example, when the assay mixture comprises “a candidate compound”, then the candidate compound(s) present in the assay mixture may all have the same structure or different structures. When the assay mixture comprises “a plurality of candidate compounds”, then the candidate compound(s) present in the assay mixture have at least two different structures.

As used herein, the term “immobilized” refers to attached to a solid surface through various means (e.g., covalent attachment, or high-affinity non-covalent attachment), in such a way that the attached moiety does not substantially diffuse into a solution (e.g., the incubation solution).

As used herein, the expression “binding site” refers to the fragment or portion of a protein, which is capable of binding to (e.g., interacting with) a compound, such as a compound identified in a method of the present disclosure, or a protein. In one embodiment, the binding site of a protein comprises a domain of the protein. In some embodiment, the binding site of a protein comprises an engineered domain of the protein.

As used herein, the term “binder compound” refers to a compound capable of targeting one or more targets (e.g., one or more proteins). In some embodiments, the compound is capable of inducing a proximity between one or more targets (e.g., one or more proteins). In some embodiments, the compound is capable of causing an effect (e.g., a degradation, change in activity, or a post-translational modification) of one or more targets (e.g., one or more POIs) in the presence of another one or more targets (e.g., E3 ligase).

As used herein, the term “targeting” refers to the capability of, or the effect or action of, associated with a target (e.g., a protein) by the referenced element (e.g., a binder compound). In some embodiments, the referenced element (e.g., the binder compound) is capable of associated with, or is associated with, the target (e.g., the protein) via a covalent attachment and/or a non-covalent attachment (e.g., a high-affinity non-covalent attachment). In some embodiments, the association between the referenced element (e.g., the binder compound) and the target (e.g., the protein) causes an effect (e.g., a degradation, change in activity, or a post-translational modification) of the target (e.g., the POI), e.g., in the presence of another target (e.g., E3 ligase or other proteins).

As used herein, the term “fragment” refers to a portion of the referenced element. For example, a fragment of a protein refers to a portion of the protein. In some embodiments, the fragment of a protein is a portion comprising one or more components of the protein (e.g., one or more components having functional and/or structural role(s) of the protein.

As used herein, the term “configured to” refers to any means (e.g., physical, or chemical, or biological) of construction or configuration of the referenced element, such that the element is capable of performing the referenced function.

As used herein, the term “complex” refers to a group of associated elements (e.g., compounds, proteins, and/or protein fragments) by various means (e.g., covalent attachment and/or non-covalent attachment). In some embodiments, the elements in the complex are associated through proximity, e.g., a proximity induced by a compound between two or more proteins or protein fragments.

As used herein, the term “affinity binder” refers to an element (e.g., a molecule, peptide, or protein moiety) that is capable of associated with the affinity component via various means (e.g., covalent attachment, or high-affinity non-covalent attachment). In some embodiments, the affinity binder is an immobilized binder that, upon contacting a complex comprising the affinity component, forms an immobilized complex (e.g., vis the association between the affinity binder and the affinity component).

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that, throughout the description, where compounds are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that, unless otherwise stated, any description of a method of treatment or prevention includes use of the compounds to provide such treatment or prevention as is described herein. It is to be further understood, unless otherwise stated, any description of a method of treatment or prevention includes use of the compounds to prepare a medicament to treat or prevent such condition. The treatment or prevention includes treatment or prevention of human or non-human animals including rodents and other disease models.

A “subject” to which administration is contemplated includes, but is not limited to, humans (i.e., a male or female of any age group, e.g., a pediatric subject (e.g, infant, child, adolescent) or an adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. In some embodiments, the subject is an agricultural entity (e.g., seeds, saplings, leaves, flowers, plants, etc).

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model. It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

As used herein, the term “preventing,” or “prevent,” describes reducing or eliminating the onset of the symptoms or complications of such disease, condition or disorder.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of humans and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

As used herein, the term “therapeutically effective amount”, refers to an amount of a pharmaceutical compound to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

It is to be understood that, for the compounds of the present disclosure capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

In some embodiments, the pharmaceutically acceptable salt is a sodium salt, a potassium salt, a calcium salt, a magnesium salt, a diethylamine salt, a choline salt, a meglumine salt, a benzathine salt, a tromethamine salt, an ammonia salt, an arginine salt, or a lysine salt.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ratio other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognize the advantages of certain routes of administration.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

A suitable pharmaceutically acceptable prodrug of a compound disclosed herein is one that is based on reasonable medical judgment as suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable prodrug of an compound disclosed herein that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxyC2-C₄ alkylamine such as 2methoxyethylamine, a phenylC₁-C₄ alkylamine such as benzylamine and amino acids such as glycine or an ester thereof.

A suitable pharmaceutically acceptable prodrug of a compound disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl.

### EXAMPLES

The following examples are provided for illustrative purposes only and are not to be construed as limiting upon the present invention.

### Example 1. Designing Exemplary Methods

Several exemplary methods are designed. The detail designs of the methods are summarized below.

### Exemplary Method 1:

The glue screening assay mixture contains the following components in a physiological buffer: GFP1-9, protein(s) of interest (POIs) tagged with another short stretch of amino acid sequence from the GFP typically corresponding to GFP11, E3 ligase(s) of choice tagged with a short stretch of amino acid sequence from a green fluorescence protein (GFP) typically corresponding to GFP10, and compound(s) of interest as a potential molecular glue. The POIs can be tagged with GFP10 instead of GFP11. In this case, the E3 ligases are tagged with GFP11. Full-length GFP consists of 11 distinct stretches of amino acid residues that form a barrel-shaped structure. GFP1-9 contains the first 9 of those distinct stretches. GFP10 indicates a stretch of amino acid residues that corresponds to the 10th of the 11 stretches in the full-length GFP. Similarly, GFP11 indicates the 11th of the 11 distinct stretches of amino acids in the full-length GFP. There are multiple variations of GFP that are either of natural origin or of genetic engineering. Despite some variations in the optimum fluorescence wavelengths, these proteins all have a similar barrel-shaped structure with 11 distinct stretches of amino acid residues. In this disclosure, GFP refers to any one of those variant forms. Other distinct stretches of amino acids can be used to tag a first protein (e.g. POIs) and a second protein (e.g. E3 ligases) as long as the two stretches that are used to tag a first protein and a second protein, respectively, are adjacent to each other in the fully formed barrel-shaped structure. In this case, GFP1-9 will be replaced with the continuous stretch of amino acid residues from the GFP excluding the two stretches that were used to tag the two proteins in such a way that the three parts of the GFP can assemble to form the barrel-shaped mature GFP. In a typical screening assay, multiple POIs up to 10 or more each with the GFP11 (or GFP10) tag can be tested simultaneously in the same assay mixture. Likewise, multiple E3 ligases up to 10 or more each with the GFP10 tag (or GFP11 when POIs are tagged with GFP10) can be tested simultaneously in the same assay mixture. GFP1-9 does not require any specific tag and is used usually at a higher concentration than any of the individual E3s or POIs. Compounds of interest can be tested one at a time. In order to increase the throughput of the screen, compounds of interest are typically pooled together into a mixture of 100-200 compounds per pool using stock solutions typically dissolved in dimethyl sulfoxide (DMSO) at a concentration of 10 mM. When 100 compounds are pooled into the mixture starting with the original 10 mM stock solutions, the pooled solution has 100 uM concentration for individual compound. When original compound stock solutions are available at higher concentrations, then higher throughput can be achieved by pooling more compounds into each pool. Alternatively, compounds can be tested at a higher concentration while maintaining the same throughput.

In addition to tagging of the POI(s) and E3(s) with GFP-derived amino acid sequences, either POI(s) or E3(s), but not both, can be modified with biotin as a handle to capture these proteins via high affinity interaction of biotin with streptavidin, avidin, or their variants. Biotinylation of the protein can be done by any number of well-established methods including biochemical conjugation using a biotin analog with a reactive functional group that forms a covalent bond with certain amino acid side chains or free N- or C-termini. Other frequently used methods include expressing the protein as a hybrid protein containing a stretch of amino acid residues that are often called “Avi tag”. The Avi tag can be biotinylated either intracellularly during protein synthesis or enzymatically after purification of the protein. Other types of tags can used in place of the biotin tag if the same tag is not found in any of other components in the assay mixture and corresponding high affinity capture reagents are available to bind the tag.

When a ternary complex is formed among any combination of POI, E3, and a glue molecule, the GFP11 and GFP10 tags on POI and E3 are brought into proximity. Under this condition, assembly of the mature GFP consisting of GFP1-9, GFP11 and GFP10 can occur at a dramatically accelerated rate compared to random assembly of these three components in solution, which can be detected by appearance of green fluorescence in the solution. The binding of the GFP1-9 to the GFP11 and GFP10 to form the barrel-shaped GFP complex is practically not reversible under physiological conditions and thus stabilizes the ternary complex of the POI, E3 and glue molecule. Appearance of fully mature GFP can be monitored by measuring development of green fluorescence in solution. This is done by repeated measurement of green fluorescence over time typically 8 hrs to overnight. Significantly higher fluorescence from certain wells compared to the majority of the wells in the plate indicates presence of a ternary complex in the well. These wells are considered as Actives.

At the end of sufficient time of incubation to allow ternary complex formation and subsequent assembly of GFP on the ternary complex, the assay mixture can be transferred to a plate pre-coated with streptavidin to capture biotin-tagged protein. The capacity of the streptavidin coated on the plate should be sufficiently high to capture all protein(s) with the biotinylated protein. When other types of tag are used in place of biotin, the plate should be pre-coated with appropriate high affinity agent in place of the streptavidin that can capture the tag. After incubation of the assay mixture in the streptavidin-coated plate, typically for 30 min to an hour, the plate is washed with a buffered solution to remove any unbound protein and compounds. When the biotin tag is put on the POI(s), most of the E3 proteins and GFP1-9 are washed away except for those that are recruited to the POI as part of the ternary complex with the glue compound. When the biotin tag is put on the E3(s), the reverse is true for POI proteins and GFP1-9. To further enhance the fluorescence signal from the fully assembled GFP, a solution containing a GFP booster reagent may be added to each well. The GFP booster is an affinity binder to GFP labeled with a fluorescent dye. The dye has the same fluorescence wavelength as the GFP with higher quantum yield. In this case, the GFP booster is a form of antibody against the fully assembled GFP that is conjugated with the chemical fluorophore with the same excitation/emission wavelength as the GFP. For example, Alexa Fluor 488-conjugated GFP nanobody from Chromotek can be used for this purpose. Due to high specificity of this reagent for fully assembled GFP over GFP1-9, GFP10 or GFP11, only those wells containing fully assembled GFP bind the GFP booster. Unbound GFP booster is washed away. To further increase the specificity of GFP booster for the fully assembled GFP, incubation time of the GFP booster solution is limited to less than 30 min. Due to the higher affinity of the GFP booster for the fully assembled GFP compared to GFP1-9, GFP10 or GFP11, shorter incubation time ensures that the GFP booster binds to the plate only when there is a fully assembled GFP captured on the plate. Alternatively, the GFP booster can be added directly to the assay mixture at the end of the incubation to form ternary complex and subsequent assembly of full length GFP but before transfer to the streptavidin-coated plate. Due to specificity of GFP booster for the full length GFP, similar results are expected. Once the plate is incubated with the GFP booster reagent and washed to remove unbound GFP booster, the plate is sealed with a transparent film and fluorescence is measured from individual wells in the plate. Other types of affinity methods can be utilized for capture of the ternary complex including use of anti-GFP antibody to capture fully assembled GFP. In addition, affinity agent can be conjugated or coated on any solid phase surface such as magnetic beads, agarose beads, or other types of beads.

The screening method can be performed multiple times. For instance, in an initial high-throughput screen, fluorescence is detected in the active wells containing the chemical diversity library (100-200 compounds per well) pooled with one or more POIs and one or more E3s. Thereafter, a second screen is conducted repeating the same pooled compounds, POI(s), and E3(s) from the initial screen in new wells. Without wishing to be bound by theory, performing the screen a second time increases confidence that any initial signals detected are repeatable and not due to noise or other circumstantial factors.

Due to the multiplexing nature of screening method, the identity of the individual POI, E3, and the glue compound in the Actives is not known at this stage. Two different methods are utilized for this identification purpose: Deconvolution and MS-based identification, which are explained below.

In Deconvolution method, individual POI, E3, and potential glue compound in the Actives are tested in such a way to uniquely and unambiguously identify which specific combination of POI, E3, and potential glue compound led to the ternary complex formation in the identified Active. In one example, all individual combinations of POI and E3 are tested in each sample using the same pool of compounds to identify the correct combination of POI and E3 first. Individual compounds are tested subsequently using the specific pair of POI and E3 that was identified in the previous step. Multiple other methods can be used as long as a unique combination of POI, E3, and the glue compound can be unambiguously identified. For example, the same protein mixture can be tested against individual compounds in the Active pool for identification of the active glue molecule with subsequent testing of individual combination of specific POI and E3 with the same active glue molecule.

In MS-based identification, the constituents in the ternary complex in the Active are identified by the standard “bottom-up mass spectrometry-based proteomics” method. In this method, the proteins captured in the well are typically reduced under denaturing condition and alkylated followed by digestion with a sequence-specific protease into smaller peptides before analysis of the samples by mass spectrometer. Multiple options are available for this well-established procedure. For example, high concentration of urea, guanidine hydrocholoride, acid-labile ionic detergent such as RapiGest or other chaotropic agents are used for the purpose of denaturation of proteins. Dithiothreitol, beta-mercaptoethanol, or TCEP (tris(2-carboxyethyl)phosphine) are often used for the purpose of reducing the disulfide bonds in the protein. Iodoacetamide, iodoacetic acid, or iodoethanol are some of the commonly used alkylating agents that covalently modify the free sulfhydryl group on the protein once the disulfide bonds are reduced. Trypsin is the most commonly used sequence-specific protease to digest proteins into peptides for the purpose of mass spectrometry-based protein analysis due to its high degree of sequence specificity for protein cleavage. Other proteases that are commonly used for this purpose include Lys-C, Arg-C and chymotrypsin. Mixture of Lys-C and trypsin or sequential use of these two proteases is also a common choice for this purpose. Amino acid sequences of the digested peptides are identified by the standard proteomics method. Briefly, this method is based on comparing the acquired m/z (mass to charge ratio) value of the peptides and their MS2 fragmentation spectra with the theoretical data obtained from in-silico digestion of the protein sequences in the protein database for the appropriate species. Numerous publications are available for this method and various software, often referred to as search engines, are readily available from multiple sources. For mass spectrometry data acquisition, in-line separation of the peptides on liquid chromatography (LC) is often combined with different modes of data acquisition by the mass spectrometer. Data-dependent MS2 acquisition (DDA) is a traditional method for this purpose, and Data-independent acquisition (DIA) method is also used. Any other data acquisition methods can be used as long as unambiguous identification of the unique peptide sequences can be established. Direct acquisition of data can be done without LC separation of the peptides especially when the sample composition is relatively simple. Matrix-assisted laser desorption/ionization (MALDI) time-of-flight instrument is one such example. Direct infusion of the digested sample through electrospray ionization interface is another example. Identity of the protein(s) in the sample can be deduced from comparison of the amino acid sequence of the peptides with those of the intact proteins in the protein database. During digestion of the proteins in the ternary complex, the molecular glue compound(s) trapped in the ternary complex are released into the digestion solution. The identity of the glue compound can be also obtained from the same sample, and often from the same experiment, by comparing the exact molecular weight of the compounds in the pool for the Actives with the expected molecular weight of the compound deduced from the m/z values of the MS1 spectra. Alternatively, the protein identity information can be obtained directly using a “top-down” method without digestion of the protein if the proteins in the ternary complex can be released into the solution. In this method, intact protein mass obtained by the mass spectrometer is compared to the expected mass of the individual proteins used in this experiment. One such method of “releasing” the intact protein from the GFP is to introduce a short peptide sequence between the E3 or POI protein and the GFP10 or GFP11 tag that can be cleaved by a highly sequence specific protease. One such example is a TEV cleavage sequence, ENLYFQS(G/A). The TEV protease cleaves between the Q and S in a highly sequence specific manner. Acidification of the sample will cause dissociation of the ternary complex. Thus, the entire protein can be released from the ternary complex bound to the plate by incubating the plate with a solution containing the TEV protease followed by acidification.

The above-described Deconvolution method and MS-based identification method are not mutually exclusive and can be used in combination for highest level of confidence for identity of individual components in the ternary complex within individual Hits. It should be also noted that the MS-based method does not provide the identity of the specific affinity-tagged protein (biotinylated E3 or biotinylated POI) that was part of the ternary complex because all biotinylated proteins will be captured from all samples regardless of presence or absence of the ternary complex formation.

### Example 2. Results for Exemplary Methods

### Results for Exemplary Method No. 1a

In one version of the assay, the glue screening assay mixture is assembled in a volume of 10 μL assay buffer (50 mM Hepes buffer, pH 7.5, 150 mM KCl, 1 mM TCEP) with 1 μg GFP1-9, 0.1 μg of GFP11-tagged RBM39, and 0.1 μg GFP10-tagged DCAF15. Two different versions of GFP11-tagged RBM39 are tested with GFP11 fused to N- and C-terminus of RBM39 (GFP11-RBM39 and RBM39-GFP11, respectively). Likewise, two different versions of GFP10-tagged DCAF15 are tested: GFP10 fused to the N-terminus of DCAF15 (GFP10-DCAF15) and GFP10 fused to the C-terminus of DCAF15 (DCAF15-GFP10). For primary screens, these four different proteins are combined and added to the plates containing compound pools. Once the Active pool is identified, the same assay mixture is tested against each individual compound within the Active pool and the confirmed compound is then tested against different pairs of E3 and POIs for deconvolution. In this example, four different assay mixtures are prepared each with different combination of GFP11-tagged RBM39 and GFP10-tagged DCAF15 (See Table A).

TABLE A

| | GFP11-RBM39 | RBM39-GFP11 |
|---|---|---|
| GFP10-DCAF15 | Combination A | Combination B |
| DCAF15-GFP10 | Combination C | Combination D |

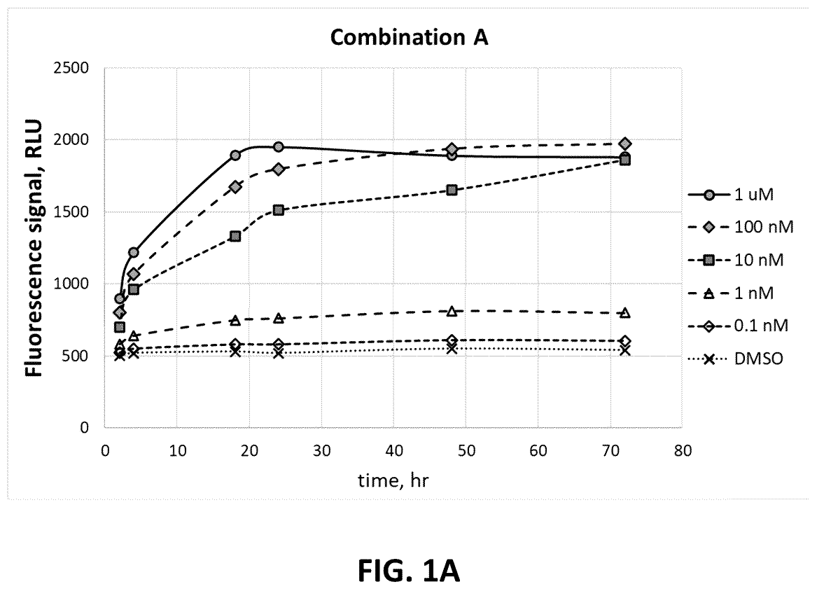

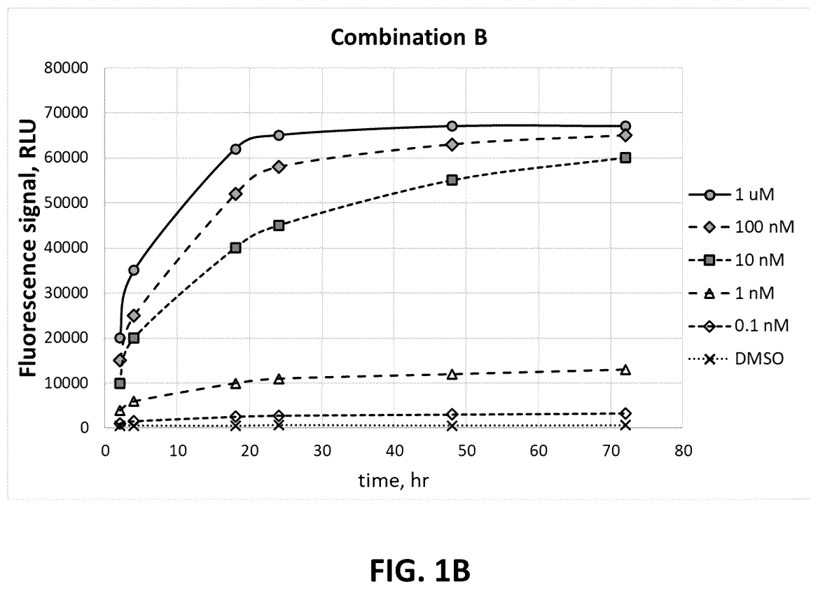

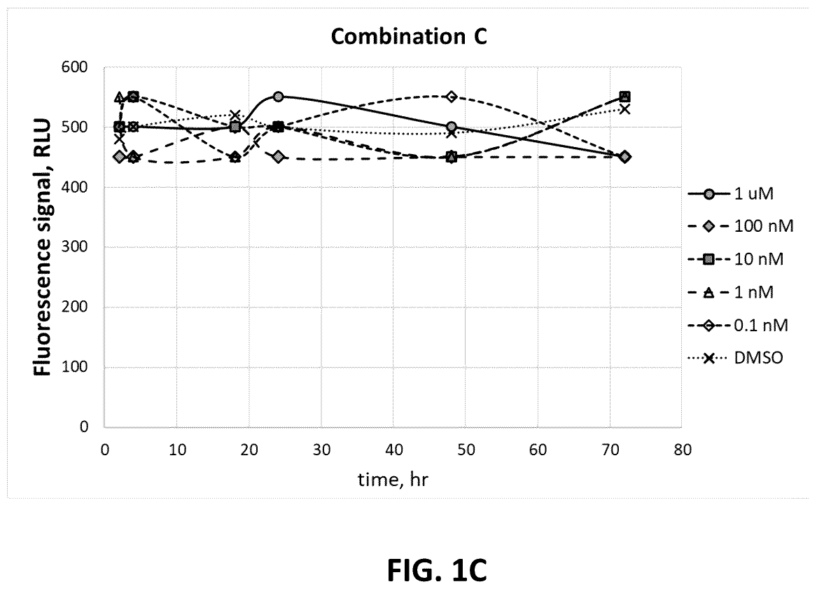

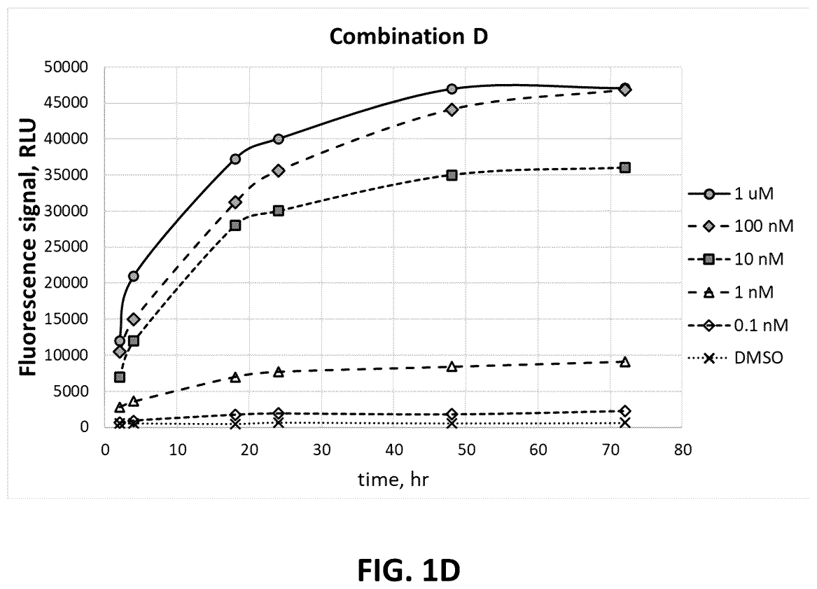

In addition to GFP11, RBM39 protein has biotin tag at the opposite side of the GFP11 via labeling of the Avi tag sequence. These different assay mixtures are added to a 384-well assay plate containing 100 nL of equimolar mixture of test compounds at various concentrations (0 nM, 10 nM, 100 nM, 1 uM, 10 uM, and 100 uM each) in DMSO. Two different test compounds are used in this experiment: indisulam (MedKoo, 201540) as a positive control and lenalidomide (aablocks, AA002FDI) as a negative control. The assay plate is sealed with a clear tape (Eppendorf 0030127838) and mixed by vortexing and spun briefly to collect the liquid at the bottom of the plate before incubation at 4° C. with shaking. Green fluorescence is measured from the assay plate after 2 hr, 6 hr, 18 hr, 24 hr, 48 hr, and 72 hr incubation using a fluorescence plate reader with excitation/emission wavelengths of 488/509 nm. FIGS. 1A-1D show simulated results with the various combinations outlined above. In FIG. 1A, FIG. 1B and FIG. 1D, dose-dependent development of signal is reaching a plateau at earlier time points with higher compound concentrations. Lower compound concentrations lead to slower development of signal and lower level of plateau. FIG. 1B represents a combination that does not show any activity.

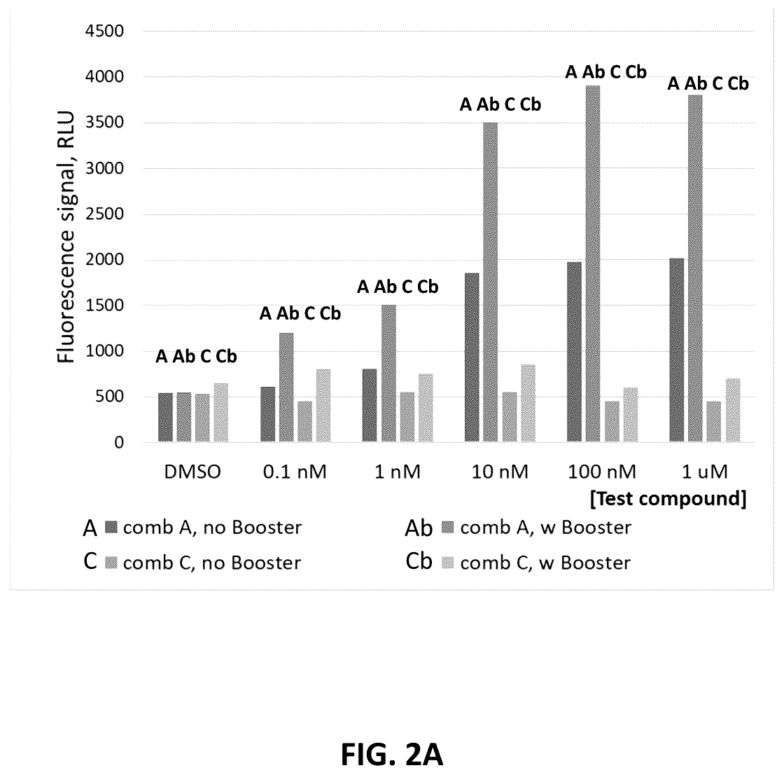

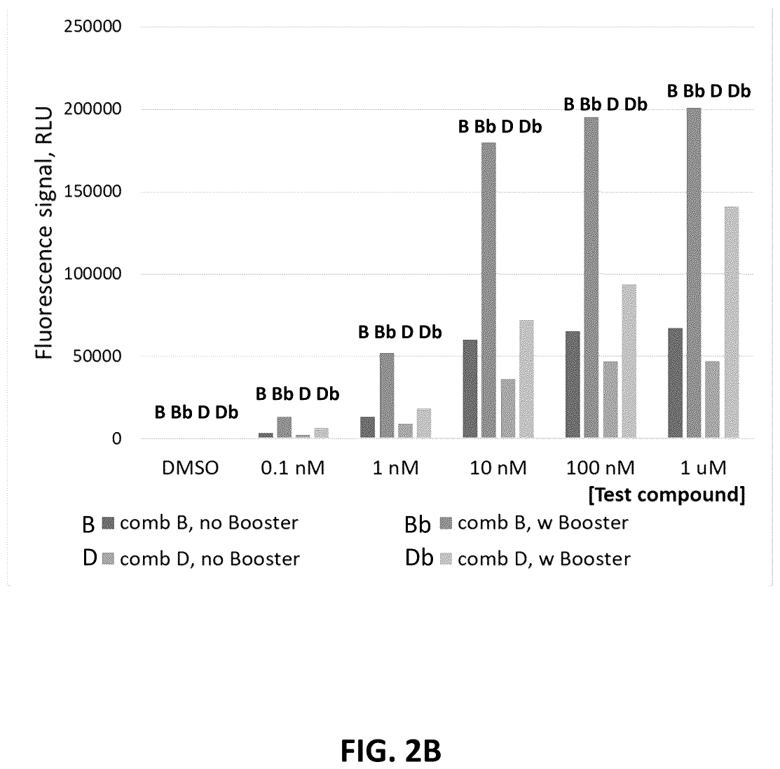

At the end of the 72 hr incubation, the contents in the assay plate are transferred to a capture plate pre-coated with streptavidin (Greiner, Cat #781995) and containing 2 μL of GFP-Booster (Chromotek gb2AF488-50) diluted 1:200 in the assay buffer. After 30 minutes of incubation at room temperature to capture biotin-tagged RBM39, the plate is placed upside down on a collection plate and spun briefly at 1500 rpm to remove the contents followed by addition of 20 μL of the assay buffer. This process is repeated three times to remove unbound proteins from the plate. Addition of the assay buffer is omitted after the third washing step, and green fluorescence is measured again as described above (simulated results in FIGS. 2A-2B) after addition of 20 μL of ammonium bicarbonate solution. Mass spectrometry-based analysis of all samples is taken without delay as described below.

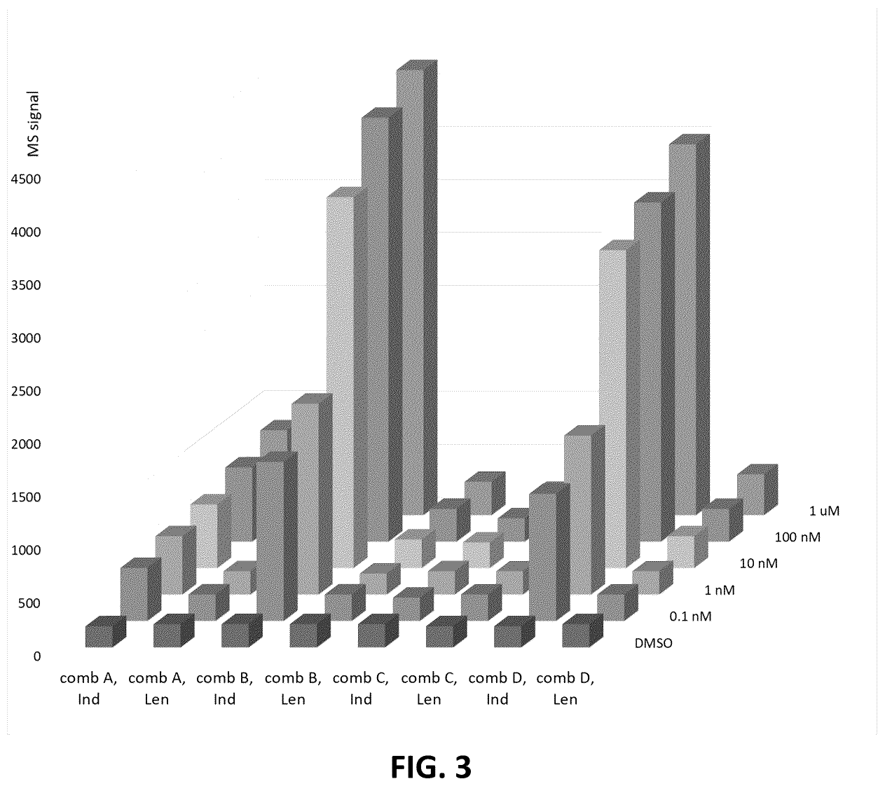

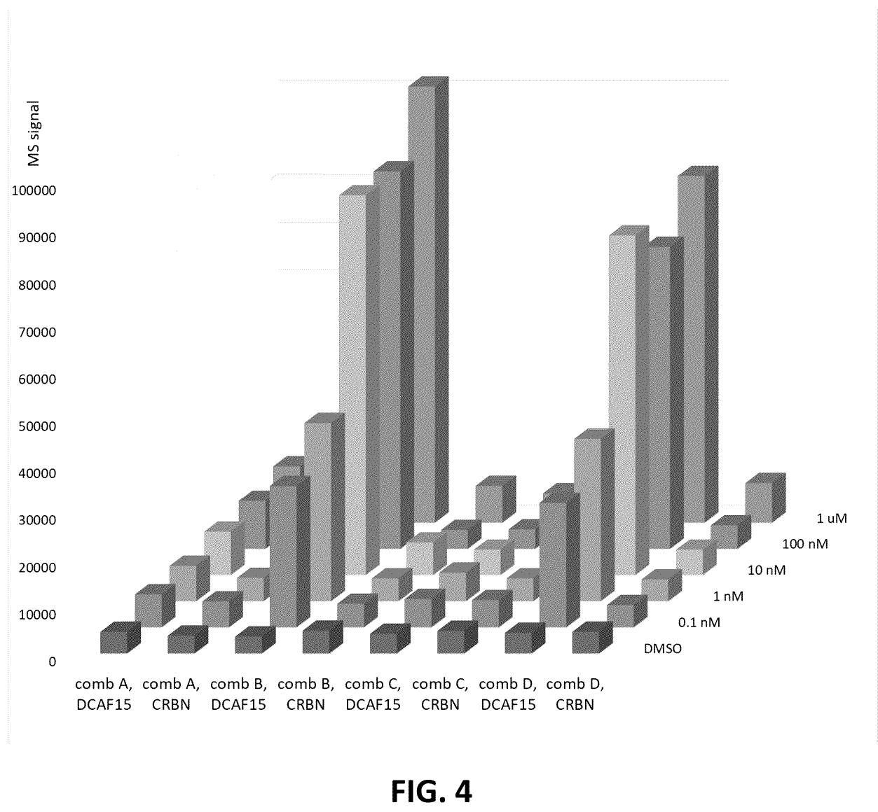

To each well in the capture plate, 5 μL of reducing agent (25 mM DTT, in 50 mM ammonium bicarbonate containing 0.05% RapiGest (Waters, 186008740)) is added and the plate is sealed with a heat-sealing tape. The capture plate is incubated at 80° C. for 5 minutes before cooling down to room temperature and transferring the contents to a new 384-well plate containing 5 μL of 75 mM iodoacetamide in 50 mM ammonium bicarbonate. The plate is incubated with shaking at room temperature in dark for 45 minutes. At the end of the incubation, 2 μL of the contents of each well are transferred to a new blank 384-well plate and mixed with 1 μL of MALDI matrix for mass spectrometry analysis of the captured glue compounds (simulated results in FIG. 3). To the remaining samples, 5 μL of trypsin solution (MS-grade from ThermoFisher, 90057 diluted to 0.05 μg/μL in in 50 mM ammonium bicarbonate) is added and the plate is sealed and incubated overnight at 37° C. At the end of incubation, 2 μL of the digested samples are transferred to another 384 well plate and mixed with 1 μL of MALDI matrix (described below) for mass spectrometry analysis of the digested peptides of the captured proteins (simulated results in FIG. 4).

MALDI matrix contains saturated solution of α-cyano-4-hydroxycinnamic acid in TA30 solvent (30:70 [v:v] acetonitrile:water with 0.1% TFA). Samples mixed with the MALDI matrix solution are incubated at 55° C. for 1 hr to facilitate hydrolysis of the RapiGest in the samples before 2 μL of the final samples are spotted onto the AnchorChip target plate (Bruker, 8280790) and dried before analysis using timsTOF mass spectrometer (Bruker) according to the manufacturer's protocol.

### Results for Exemplary Method No. 1b

In another version of the assay, the glue screening assay mixture is assembled in a volume of 2 μL assay buffer (50 mM HEPES buffer, pH 7.5, 150 mM KCl, 4 mM DTT, 0.1 mg/mL BSA, 0.25 mM octyl-beta-glucopyranoside, 5% glycerol with 0.3 μg GFP1-9, 0.05 μg of GFP11-tagged IKZF2, and 0.3 μg GFP10-tagged CRBN. Various variations of the buffer provide consistent signal in this assay (0.1-1 mg/mL BSA, 0-10% glycerol, 0-1 mM octyl-beta-glucopyranoside). Similar to the previous version, two different versions of GFP11-tagged IKZF2 are tested with GFP11 fused to N- and C-termini of IKZF2 (GFP11-IKZF2 and IKZF2-GFP11, respectively). Likewise, two different versions of GFP10-tagged CRBN are tested: GFP10 fused to the N-terminus of CRBN (GFP10-CRBN) and GFP10 fused to the C-terminus of CRBN (CRBN-GFP10). Concentrations correspond to 5 μM of GFP (1-9), 1.25 μM of each of the IKZF2 constructs and 0.5 μM of each of the two CRBN constructs. Concentrations of the IKZF2 and CRBN proteins were optimized for best signal to background by titrating the proteins against each other.

For primary screens, these five different proteins (GFP1-9, GFP11-IKZF2, IKZF2-GFP11, GFP10-CRBN, and CRBN-GFP10) are combined in the assay buffer and added to the plates containing different compound pools. During deconvolution, assay mixture is tested against each individual compound and the confirmed compound is then tested against different pairs of POI and E3.

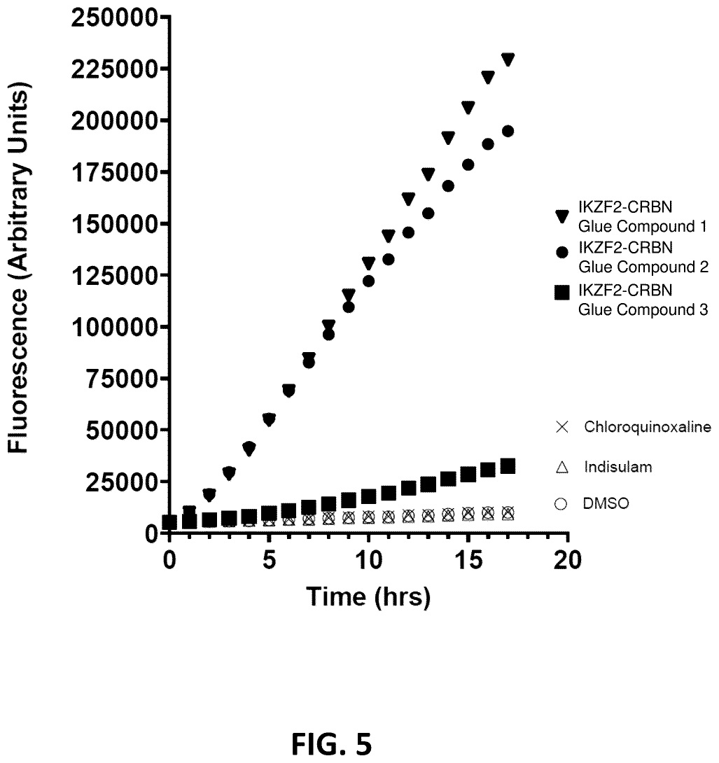

Upon the addition of the protein mix to compounds known to be established glues (IKZF2-CRBN Glue Compound 1, IKZF2-CRBN Glue Compound 2, IKZF2-CRBN Glue Compound 3) for IKZF2-CRBN, GFP signal increased over time compared to the DMS control (FIG. 5). The signals generated in these experiments are specific to these IKZF2-CRBN glues, because the signal from other compounds (Chloroquinoxaline, Indisulam) were indistinguishable from the DMSO control. These compounds are known glues for the protein pair of RBM39 and DCAF15 and are known to be devoid of glue activity for IKF2-CRBN protein pair (FIG. 5).

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A method of identifying a first protein, a second protein, a compound targeting the first protein and the second protein, or any combination thereof, comprising:
(i) providing an assay mixture comprising:
(a) a first protein or a fragment thereof, covalently attached to a first tag GFP fragment;
(b) a second protein or a fragment thereof, covalently attached to a second tag GFP fragment;
(c) a detector GFP fragment complementary to the first tag GFP fragment and the second tag GFP fragment; and
(d) a candidate compound,
wherein the first tag GFP fragment, the second tag GFP fragment, and the detector GFP fragment are configured to generate or enhance an assay signal upon the assay mixture resulting in a complex comprising the first protein or fragment thereof, the second protein or fragment thereof, and the compound; and
(ii) identifying the first protein, the second protein, the compound, or any combination thereof associated with the complex, wherein the identified compound is capable of causing the degradation of the first protein, in the presence of the second protein.

2. A method of identifying a first protein, a second protein, a compound targeting the first protein and the second protein, or any combination thereof, comprising:
(i) providing an assay mixture comprising:
(a) a first protein or a fragment thereof, covalently attached to a first tag GFP fragment;
(b) a second protein or a fragment thereof, covalently attached to a second tag GFP fragment;
(c) a detector GFP fragment complementary to the first tag GFP fragment and the second tag GFP fragment; and
(d) a candidate compound,
wherein the first protein or fragment thereof, and/or the second protein or fragment thereof, is covalently attached to an affinity component; and
wherein the first tag GFP fragment, the second tag GFP fragment, and the detector GFP fragment are configured to generate or enhance an assay signal upon the assay mixture resulting in a complex comprising the first protein or fragment thereof, the second protein or fragment thereof, and the compound;
(ii-a) contacting the complex with an immobilized affinity binder targeting the affinity component, thereby forming an immobilized complex; and
(ii-b) detecting and/or isolating the immobilized complex, thereby identifying the first protein, the second protein, the compound, or any combination thereof, wherein the identified compound is capable of causing the degradation of the first protein, in the presence of the second protein.

3. A method of identifying a first protein, a second protein, a compound targeting the first protein and the second protein, or any combination thereof, comprising:
(i) providing an assay mixture comprising:
(a) a first protein or a fragment thereof, covalently attached to a first tag GFP fragment;
(b) a second protein or a fragment thereof, covalently attached to a second tag GFP fragment;
(c) a detector GFP fragment complementary to the first tag GFP fragment and the second tag GFP fragment; and
(d) a candidate compound,
wherein the first tag GFP fragment, the second tag GFP fragment, and the detector GFP fragment are configured to generate or enhance an assay signal upon the assay mixture resulting in an induced proximity between the first protein or fragment thereof, and the second protein or fragment thereof; and
(ii) identifying the first protein, the second protein, the compound, or any combination thereof associated with the induced proximity, wherein the identified compound is capable of causing the degradation of the first protein, in the presence of the second protein.

4. (canceled)

5. The method of claim 1, wherein the assay mixture comprises a plurality of different first proteins or fragments thereof.

6. The method of claim 1, wherein the assay mixture comprises a plurality of different second proteins or fragments thereof.

7. The method of claim 1, wherein the assay mixture comprises a plurality of different candidate compounds.

8. The method of claim 1, wherein the assay mixture results in the complex comprising the first protein or fragment thereof, the second protein or fragment thereof, and the candidate compound.

9. The method of claim 1, wherein the assay signal is fluorescence.

10.-12. (canceled)

13. The method of claim 1, wherein the first tag GFP fragment and the second tag GFP fragment independently comprises one or more of GFP1, GFP2, GFP3, GFP4, GFP5, GFP6, GFP7, GFP8, GFP9, GFP10, and GFP11.

14. The method of claim 1, wherein the first tag GFP fragment and the second tag GFP fragment independently comprises one or more of GFP10 and GFP11.

15. The method of claim 1, wherein the first tag GFP fragment comprises GFP10, and the second tag GFP fragment comprises GFP11.

16. The method of claim 1, wherein the detector GFP fragment is selected from GFP1, GFP2, GFP3, GFP4, GFP5, GFP6, GFP7, GFP8, and GFP9.

17. The method of claim 1, wherein the first protein is a protein of interest (POI).

18. The method of claim 1, wherein the second protein is a ubiquitin ligase.

19. The method of claim 1, wherein step (i) further comprises detecting the generated or enhanced assay signal.

20.-27. (canceled)

28. A method of degrading a first protein or a second protein in a subject, comprising administering to the subject a compound identified by the method of claim 1.

29. A method of modulating a PPI between a first protein and a second protein in a subject, comprising administering to the subject a compound identified by the method of claim 1.

30. A method of treating and/or preventing a disease or disorder associated with a first protein or a second protein in a subject, comprising administering to the subject a therapeutically effective amount of a compound identified by the method of claim 1.
